SEQUENCE LISTING (1 sequence)

SEQ ID NO: 1 - DNA, 60 bp, Artificial Sequence
  synthetic sequence
gggcccaccgcgtacgacgccggcggccttcacctggatgcagtacgtccagtcagccat

(19) United States
(12) Patent Application Publication
(10) Pub. No.: US 2019/0385697 A1
(43) Pub. Date: Dec. 19, 2019

(54) METHODS FOR PREDICTING TRANSCRIPTION FACTOR ACTIVITY

(71) Applicant: The Regents of the University of Colorado, A Body Corporate, Denver, CO (US)

(72) Inventors: Robin DOWELL-DEEN, Broomfield, CO (US); Joseph AZOFEIFA, Boulder, CO (US); Mary A. ALLEN, Broomfield, CO (US)

(21) Appl. No.: 16/485,717

(22) PCT Filed: Feb. 14, 2018
(86) PCT No.: PCT/US18/18230

§ 371 Date: Aug. 13, 2019

Related U.S. Application Data
(60) Provisional application No. 62/458,572, filed on Feb. 14, 2017.

(51) Int. Cl. G16B 20/30; G01N 33/50; C12Q 1/6869; G16B 40/10; G16B 50/30; G06F 17/18

(52) U.S. Cl. CPC G16B 20/30; G01N 33/50; G06F 17/18; G16B 40/10; G16B 50/30; C12Q 1/6869

(57) ABSTRACT

Provided herein are methods for approximating transcription factor (TF) activity in a cell. The methods can approximate changes in TF activity resulting from a stimulus, such as a drug or cell differentiation. Some methods for approximating TF activity in a cell are laboratory methods. Some methods may be used to identify diagnostic signatures of transcription factor activity, and identify cell type or disease state. Computer-based systems for evaluating the effect of a stimulus on TF activity in a cell are also provided.

### CROSS REFERENCE TO RELATED APPLICATIONS

This International Patent Application claims the benefit of U.S. Provisional Patent Application No. 62/458,572, filed Feb. 14, 2017, the disclosure of which is incorporated herein by reference in its entirety.

### STATEMENT OF GOVERNMENT SUPPORT

This invention was made with government support under grant numbers DGE1144807 and DBI1262410 awarded by the National Science Foundation. The government has certain rights in the invention.

### SEQUENCE LISTING

The instant application contains a Sequence Listing which has been submitted via EFS-Web and is hereby incorporated by reference in its entirety. The ASCII copy, created on Feb. 13, 2018, is named 466888.60_SL_ST25.txt, and is 4,096 bytes in size.

### BACKGROUND

Transcription is orchestrated by the sequence-specific binding of transcription factors (TFs) to DNA, resulting in regulation of gene expression programs. Hence, TFs function as major determinants of cell state. Despite their critical importance for controlling cellular phenotypes, no reliable method for ascertaining global TF activity in a cell exists to date.

Chromatin immunoprecipitation (ChIP) studies have identified binding sites for many of the approximately 1,400 transcription factors encoded within the human genome, allowing estimation of a DNA binding motif model for more than 600 factors. However, studies comparing TF binding events to RNA expression levels have revealed that many TF binding sites have no apparent effect on nearby transcription. Distinguishing such “silent” TF binding events from those with regulatory capacity is a fundamental challenge.

Identifying “active” TFs (as opposed to “silent” TFs) in a cell is challenging. Because binding (measured by ChIP) does not equate with transcriptional regulatory activity, the most common alternative leverages changes in gene expression upon perturbation of the TF, where perturbation includes knockdowns, knockouts, over-expression, or chemical stimulation. Additionally, because expression studies (typically by RNA-seq) are steady state assays, this approach assays expression at long time points after the perturbation or stimulus. Hence the changes in expression observed are a mix of primary effects and secondary (cellular adaptation) responses. Consequently, expression based methods have poor signal-to-noise characteristics. Furthermore, attempts to infer TF activity from patterns of TF motif instances at annotated protein coding genes has been limited by the fact that most TF binding occurs within regions of the genome distal to protein coding genes, making the pattern of TF motifs at protein coding genes a poor indicator of TF activity. Finally, the length and duration of expression-based approaches could be particularly prohibitive to a fuller understanding of cell activity, as perturbations to the cell could alter numerous aspects of cellular physiology, resulting in an inaccurate identification of active TFs.

Furthermore, both approaches (ChIP and expression) require individually measuring the activity level of each TF. As a result, such measurements were slow and cumbersome and provided only limited information regarding the cell state. In particular, these prior approaches were able to effectively analyze only a few TFs within a given window of time and resources (e.g., in the order of 10s of TFs), which were significantly fewer than the approximately 600 TFs available on a more global level for which DNA binding motif models are available. In other words, the time and resources needed to analyze teach TF on an individual basis, per the prior approach, prohibited the effective analysis of the larger TF spectrum for a cell.

Most TF binding, however, occurs within regions of the genome distal to protein coding genes. These binding events often correspond to enhancer regions known to be important for regulation of gene expression and cellular identity. Active enhancers are often characterized by the presence of short, unstable, bidirectional transcripts termed enhancer RNAs (eRNAs). Importantly, when a specific activator TF is activated, eRNA transcription generally increases over at the location of the TF binding event. Whereas activation of a repressor TF results in a decrease in eRNA transcription over the location of the binding event. However, eRNA detection requires extremely sensitive methods, both in the laboratory as well as computationally. Because they are unstable, eRNAs are rarely observed via steady state RNA assays such as RNA-seq. Nascent transcription assays capture all transcription throughout the genome, regardless of transcript stability. Hence nascent assays capture eRNA transcription. The functions of eRNAs are only beginning to be understood.

Several methods provide for the identification of enhancers. However, identification of active enhancers does not equate to active transcription factors. Enhancers are densely populated with TF recognition motifs and show signals in ChIP for a large number of TFs.

To address these and other shortcomings of prior approaches, the instant disclosure provides improved techniques for analyzing TF activity in a cell that can better account for TF activity in a cell from a global perspective (e.g., with respect to hundreds or a thousand TFs, rather than only a few) in a faster and more efficient manner using only nascent transcription data. By providing an analysis on a global perspective using cell-specific transcription data, these improved techniques enable a fuller understanding of the effects of perturbations on a cell. Furthermore, certain embodiments can lead to more effective medical treatments because the active TFs can be more readily ascertained and targeted, e.g., through TF-specific compounds.

For example, some embodiments generate a genome-wide nascent transcription profile for the cell. These embodiments then model transcription factor activity in the cell using enhancer RNA (eRNA) origination sites in the cell's genomic DNA, DNA binding motif instances for at least one transcription factor in the cell's genomic DNA, and measured distanced from each of the identified DNA binding motif instances to at least one of the eRNA origination sites. In particular, these embodiments create a Motif-Displacement (MD) model to approximate TF activity in the cell. Additional details regarding the MD model and its applications are provided below.

### SUMMARY

In a first aspect, described herein is a laboratory method for evaluating the effect of a stimulus on a cell using a Motif-Displacement (MD) model that approximates transcription factor activity in the cell, the method comprising: a) locating a first set of enhancer RNA (eRNA) origination sites in the cell's genomic DNA using a first genome-wide nascent transcription profile for the cell; b) identifying DNA binding motif instances for transcription factors in the cell's genomic DNA; c) for each eRNA origination site in the first set of eRNA origination sites, measuring a number of DNA binding motif instances for each of the transcription factors occurring within a first radius of the eRNA origination site and measuring a number of DNA binding motif instances for each of the transcription factors occurring within a second radius of the eRNA site, wherein the first radius and the second radius are each centered at each of the eRNA origination sites of the first set of eRNA origination sites, and the second radius is greater than the first radius; d) calculating, using one or more processors executing instructions stored in a tangible, non-transitory storage medium, a first MD-level for each of the transcription factors based on the number of DNA binding motif instances for that transcription factor occurring within the first radius of the eRNA origination sites of the first set of eRNA origination sites and the number of DNA binding motif instances for that one transcription factors occurring within the second radius of the eRNA origination sites of the first set of eRNA origination sites; e) applying a stimulus to the cell; f) locating a second set of eRNA origination sites in the cell's genomic DNA using a second genome-wide nascent transcription profile for the cell, wherein the second genome-wide nascent transcription profile is generated after applying the stimulus to the cell; g) for each eRNA origination site in the second set of eRNA origination sites, measuring a number of DNA binding motif instances for each of the transcription factors occurring within the first radius of the eRNA origination site and measuring a number of DNA binding motif instances for each of the transcription factors occurring within the second radius of the eRNA site; h) calculating, using one or more processors executing instructions stored in a tangible, non-transitory storage medium, a second MD-level for each of the transcription factors based on the number of DNA binding motif instances for that transcription factor occurring within the first radius of the eRNA origination sites of the second set of eRNA and the number of DNA binding motif instances for that one transcription factors occurring within the second radius of the eRNA origination sites of the second set of eRNA origination sites; and i) approximating effects of the stimulus on the transcription factor activity in the cell by identifying biologically significant differences between the first MD-level and the second MD-level.

In some embodiments, the laboratory method further comprises generating at least one of the first genome-wide nascent transcription profile for the cell and the second genome-wide nascent transcription profile.

In a second aspect, described herein is a computer-based system for evaluating the effect of a stimulus on a cell using a Motif-Displacement (MD) model that approximates transcription factor activity in a cell, the system comprising: one or more processors; and a non-transitory, tangible storage medium containing instructions that, when executed by the processor, cause the one or more processors to: a) locate a first set of enhancer RNA (eRNA) origination sites in the cell's genomic DNA using a first genome-wide nascent transcription profile for the cell; b) identify DNA binding motif instances for transcription factors in the cell's genomic DNA; c) for each eRNA origination site in the first set of eRNA origination sites, measuring a number of DNA binding motif instances for each of the transcription factors occurring within a first radius of the eRNA origination site and measuring a number of DNA binding motif instances for each of the transcription factors occurring within a second radius of the eRNA site, wherein the first radius and the second radius are each centered at each of the eRNA origination sites of the first set of eRNA origination sites, and the second radius is greater than the first radius; d) calculate a first MD-level for each of the transcription factors based on the number of DNA binding motif instances for that transcription factor occurring within the first radius of the eRNA origination sites of the first set of eRNA origination sites and the number of DNA binding motif instances for that one transcription factors occurring within the second radius of the eRNA origination sites of the first set of eRNA origination sites; e) locate a second set of eRNA origination sites in the cell's genomic DNA using a second genome-wide transcription profile for the cell, wherein the second genome-wide nascent transcription profile is generated after applying a stimulus to the cell; f) for each eRNA origination site in the second set of eRNA origination sites, measuring a number of DNA binding motif instances for each of the transcription factors occurring within the first radius of the eRNA origination site and measuring a number of DNA binding motif instances for each of the transcription factors occurring within the second radius of the eRNA site, g) calculate a second MD-level for each of the transcription factors based on the number of DNA binding motif instances for that transcription factor occurring within the first radius of the eRNA origination sites of the second set of eRNA origination sites and the number of DNA binding motif instances for that one transcription factors occurring within the second radius of the eRNA origination sites of the second set of eRNA origination sites; and h) approximate effects of the stimulus on the transcription factor activity in the cell by identifying biologically significant differences between the first MD-level and the second MD-level.

In a third aspect, described herein is a method for identifying active transcription factors in a cell, the method comprising: a) locating enhancer RNA (eRNA) origination sites in the cell's genomic DNA by analyzing a genome-wide nascent transcription profile for the cell; b) identifying DNA binding motif instances for transcription factors in the cell's genomic DNA; c) measuring a number of DNA binding motif instances for each of the transcription factors occurring within a first radius of each of the eRNA origination sites; d) measuring a number of DNA binding motif instances for each of the transcription factors occurring within a second radius of each of the eRNA origination sites, wherein the first radius and the second radius are each centered at each of the eRNA origination sites and wherein the second radius is greater than the first radius; e) using one or more processors to determine a Motif-Displacement (MD) level that approximates transcription factor activity in the cell, the processor executing instructions stored in a tangible, non-transitory storage medium in order to: e1) calculate an observed MD-level for each of the transcription factors using the number of DNA binding motif instances for a transcription factor occurring within the first radius of the eRNA origination site and the number of DNA binding motif instances for that transcription factor occurring within the second radius of the eRNA origination site; e2) calculate an expected MD-level for each of the transcription factors; and e3) allocate each of the transcription factor as active in the cell if the calculated observed MD-level is greater than the expected MD-level and if the difference between the calculated MD-level and the expected MD-level is biologically significant.

In some embodiments, the method for identifying active transcription factors in a cell further comprises the step of identifying one or more compounds that are biologically effective with respect to the active transcription factors.

In some embodiments, the method for identifying active transcription factors in a cell further comprises generating the genome-wide nascent transcription factor profile.

In some embodiments described herein, the stimulus is a drug, a biologic, a compound or combination of compounds capable of initiating cellular differentiation or causing a disease state; an environmental stress, time, or a combination thereof.

In some embodiments, a genome-wide nascent transcription profile is generated by a technique selected from: global run-on sequencing (GRO-seq), global run-on cap sequencing (GRO-cap), chromatin immunoprecipitation sequencing (ChIP-seq), precision nuclear run-on sequencing (PRO-seq), cap analysis of gene expression with deep sequencing (CAGE), 5′-end serial analysis of gene expression (SAGE), native elongating transcript sequencing (NET-seq), chromatin isolation by RNA purification (ChIRP-seq), assay for transposase-accessible chromatin with high throughput sequencing (ATAC-seq), transient transcriptome sequencing (TT-seq), chromatin run-on sequencing (ChRO-seq) and bromouridine UV sequencing (BruUV-seq).

In some embodiments, a set of eRNA origination sites is located utilizing one of: Tfit, dREG, groHMM, Vespucci, and FStitch.

In some embodiments, the first radius is selected from between 50 base-pairs and 300 base-pairs. In some embodiments, the first radius is 150 base-pairs.

In some embodiments, the second radius is selected from between 500 base-pairs and 3000 base-pairs. In some embodiments, the second radius is 1500 base-pairs.

In some embodiments, the second radius is 7 to 13 times larger than the first radius. In some embodiments, the second radius is 10 times larger than the first radius.

In some embodiments, the first radius is 150 base-pairs and the second radius is 1500 base-pairs.

In some embodiments, transcription factor activity for a given transcription factor is approximated as increased if the second MD-level is greater than the first MD-level, approximated as decreased if the second MD-level is smaller than the first MD-level, or approximated as unchanged if the second MD-level approximately equals the first MD-level.

### BRIEF DESCRIPTION OF THE DRAWINGS

The patent or application file contains one or more drawings executed in color and/or one or more photographs.

FIG. 1A is a representation of an example locus displaying nascent transcript sequencing read coverage with the overlain density estimation via Tfit and the associated eRNA origin predictions.

FIG. 1B represents a genome-wide meta-signal for marks of active chromatin aligned to eRNA origins inferred by Tfit.

FIG. 1C represents the overlap of eRNA origins (columns) with regulatory protein (rows) binding data (measured by ChIP).

FIG. 1D is a histogram representing the spatial displacement of the TF binding peak from eRNA origins, rows correspond to the same proteins in FIG. 1C.

FIG. 1E is a swarm plot displaying the number of bound TFs at sites of open chromatin grouped by eRNA association.

FIG. 1F is a pairwise co-association map, where increased heat indicates a greater proportion of TF binding sites also bound by another TF.

FIG. 2 is a representation of an annotated super enhancer region. Green dots indicate the eRNA origin estimate.

FIG. 3 is a histogram representing the association of bidirectional transcription sites with promoter regions.

FIG. 4 is a bar graph representing the overlap between sites of non-promoter associated bidirectional transcription and marks of regulatory DNA.

FIG. 5A is a histogram representing the proportion of eRNAs associated with the binding sites for a given transcription factor.

FIG. 5B is a histogram representing the number of unique TF binding peaks occurring at individual eRNAs.

FIG. 5C is a histogram representing the distribution of estimated peak displacements of TF from eRNA.

FIG. 6A is a histogram representing the fraction of TF binding events associated with an eRNA.

FIG. 6B is a box-and-whiskers plot displaying the median/variability in TF binding sites associated with a variety of histone marks associated with enhancers.

FIGS. 6C-6E compares transcription at target genes (of the enhancer) for TF binding sites that differ between two cell types only in the presence (or absence) of eRNAs.

FIG. 7 is a histogram of p-values following the test of the hypothesis that TF binding sites associated with cell type unique eRNAs modulate local gene expression.

FIG. 8A is a pair of heatmaps displaying the frequency of TF binding motif instances centered at the eRNA origins predicted by Tfit from a K562 GRO-cap (SRR1552480) experiment for sites bound and not bound by ChIP.

FIG. 8B is a histogram representing the distribution of estimated RNAP footprint size obtained from Tfit.

FIG. 8C is a dot plot showing that MD-levels are higher in regions bound by a TF compared to those not bound.

FIG. 9A is an example locus of GRO-seq (nascent transcription) overlaid with a diagram depicting the inferred eRNA origin and the motif offsets used to calculate the MD-level, which is also given (SEQ ID NO: 1).

FIG. 9B is a heatmap representing the TF motif displacement distribution for all TF motif models within HOCOMOCO in a single nascent transcription dataset. The calculated MD-levels are plotted on the right.

FIG. 9C is a dot plot representing a comparison between the expected MD-level for a motif model and the observed MD-level.

FIG. 9D is a color map representing fluctuations in eRNA and TF motif co-localization across experiments in different cell types.

FIG. 9E is a pair of heat maps showing representative TF motif displacements and the corresponding MD-level for two TF motif models (JUND and CLOCK).

FIGS. 10A-10B indicate the genome-wide bias in ACTG frequency at eRNA origins.

FIG. 11A is a heat map representing the variance in MD-levels for the database of motif models (rows) across a large compendia of human nascent transcription datasets.

FIG. 11B is a heat map representing relative MD-levels for each TF motif model (rows) across a large compendium of human nascent transcription datasets (columns).

FIGS. 12A-12C indicate the motif displacement distribution, MD-level, and number of motifs within 1.5 KB of any eRNA origin before and after stimulation (top panels), and (bottom panel) the change in MD-level following perturbation (Y-axis) for all motif models relative to the number of motifs within 1.5 KB of any eRNA origin (X-axis) for TP53 (FIG. 12A), NFKB1 (FIG. 12B), and ESR1 (FIG. 12C).

FIG. 12D shows significant changes in MD-level across a time series dataset following treatment with Flavopiridol.

FIG. 12E shows significant changes in MD-level across a time series dataset following treatment with Kdo2-lipid A (KLA).

FIGS. 13A-13C are dot plots presenting MD-level comparisons for promoter-associated bidirectional transcripts between treatment/control pairs for Nutlin-3a (FIG. 13A), TNFα (FIG. 13B) and estradiol (FIG. 13C).

FIGS. 14A-14C are dot plots presenting MD-level comparisons between biological replicates for Nutlin-3a (FIG. 14A), TNFα (FIG. 14B) and estradiol (FIG. 14C).

FIG. 15 is a table indicating TF motif enrichment proximal to treatment unique eRNAs.

FIG. 16A is a plot representing MD-level change following differentiation of human embryonic stem cells to pancreatic endoderm.

FIGS. 16B-16C are dot plots representing change in MD-level between K562 cells and GM12878 cells (FIG. 16B) and between lung cells and lung carcinoma cells (FIG. 16C).

FIG. 16D provides a comparison of MD-levels for individual TFs between all possible pairs of datasets.

FIG. 16E represents a co-association network of TF factors (blue nodes) and cell type (green nodes).

FIG. 17A is a distance matrix where each cell's heat is proportional to the number of significantly different MD-levels.

FIG. 17B indicates dimensionality reduction by t-Distributed Stochastic Neighbor Embedding (TSNE) of the distance matrix in FIG. 17A.

FIG. 18A is a heatmap, where each cell indicates the average number of significantly altered MD-levels (p-value<10⁻⁶) between any two experiments that are annotated as the associated cell type.

FIG. 18B presents the distribution of the number of significantly different MD-levels grouped by comparison type: same (e.g. ESC to ESC) or different (e.g. HeLa vs LnCAP) cell type.

FIG. 19 illustrates components of a processor-based laboratory system, some or all of which may be used in various embodiments discussed herein.

While the disclosed subject matter is amenable to various modifications and alternative forms, specific embodiments are described herein in detail. The intention, however, is not to limit the disclosure to the particular embodiments described. On the contrary, the disclosure is intended to cover all modifications, equivalents, and alternatives falling within the scope of the disclosure as defined by the appended claims.

Similarly, although illustrative methods may be described herein, the description of the methods should not be interpreted as implying any requirement of, or particular order among or between, the various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As the terms are used herein with respect to ranges, “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

### DETAILED DESCRIPTION

Certain embodiments described herein provide methods for predicting transcription factor (TF) activity in a cell. In some embodiments, the methods can predict changes in TF activity resulting from a stimulus, such as a drug or cell differentiation. In other embodiments, the methods may be used to identify diagnostic signatures of transcription factor activity, and identify cell type or disease state. As discussed below in more detail, at least some steps of these methods can be implemented using a processor executing software stored in a tangible, non-transitory storage medium. For example, the software can be stored in the long-term memory (e.g., solid state memory) in a genetic sequencer, executed by the processor in the genetic sequencer. In other embodiments, the software can be stored in a separate system configured to access sequencing information from a genetic sequencer.

Despite being critical to understanding transcriptional regulation, there is no reliable method for measuring global (i.e., all) transcription factor activity in cells. Experimental approaches, such as chromatin immunoprecipitation (ChIP) and TF perturbation experiments (knock-out/-down) followed by expression analysis may be used to attempt to identify transcription factor activity. With ChIP analysis, binding sites for a single TF are identified, while knock-out experiments measure affected gene expression after elimination or deactivation of one or several TF. However, these methods have significant drawbacks, including limited throughput, that binding of a TF to the promoter of a gene does not necessarily indicated TF activity as post-translational modifications may be required for TF activity, multiple TFs may regulate a single gene and binding does not guarantee gene regulatory activity, and it may not be clear whether observed changes result from the knocked-out TF or some other effect. Changes in steady state measurements of expression reflect not only the primary effects of the transcription factor, but also secondary effects of the regulatory network.

TFs exert their regulatory influence through the binding of enhancers, resulting in coordination of gene expression programs. Active enhancers are often characterized by the presence of short, unstable transcripts call enhancer RNAs (eRNAs). While their function remains unclear, the studies described herein demonstrate that eRNAs offer a powerful readout of TF activity. As described herein, sites of eRNA origination are inferred across hundreds of publicly available nascent transcription data sets. The eRNAs are demonstrated to initiate from sites of TF binding. By quantifying the co-localization of TF binding motif instances and eRNA origin sites, a statistic capable of inferring TF activity is derived. This approach provides a fundamentally unique strategy for predicting TF activity.

Certain embodiments provide methods for predicting transcription factor activity in a cell. In some embodiments, the method includes i) identifying eRNA origination sites in the cell's genomic DNA by analyzing a genome-wide nascent transcription profile, ii) identifying DNA binding motif instances for a TF in the cell's DNA, iii) measuring the number of DNA binding motif instances for each transcription factor occurring within a first radius (h) radius of each of the eRNA origination sites, iv) measuring the number of DNA binding motif instances for each transcription factor occurring within a second radius (H) of each of the eRNA origination sites, where the first radius and the second radius are each centered at the eRNA origination sites, and the second radius is greater than the first radius, v) generating a motif-displacement (MD) model, including calculating an MD-level for each individual TF, vi) calculating an expected MD-level for each individual TF, and v) predicting a TF to be active in the cell if the TFs calculated MD-level is greater than the expected MD-level for that TF.

In some embodiments, the provided methods predict global TF activity in a cell. That is, TF activity for all TFs for which a TF DNA binding motif model is known. In some embodiments, the provided methods predict TF activity of a subset of TFs for which a TF DNA binding motif model is known. In some embodiments, the provided methods predict TF activity of at least 600 TFs.

In some embodiments, the methods for predicting transcription factor activity in a cell also include generating a genome-wide nascent transcription profile for the cell. Several methods for generating a genome-wide nascent transcription profile are known in the art. These include but are not limited to global run-on sequencing (GRO-seq), GRO-cap, chromatin immunoprecipitation sequencing (ChIP-seq), precision nuclear run-on sequencing (PRO-seq), cap analysis of gene expression with deep sequencing (CAGE), 5′-end serial analysis of gene expression (SAGE), native elongating transcript sequencing (NET-seq), chromatin isolation by RNA purification (ChIRP-seq), assay for transposase-accessible chromatin with high throughput sequencing (ATAC-seq), transient transcriptome sequencing (TT-seq), chromatin run-on sequencing (ChRO-seq), and bromouridine UV sequencing (BruUV-seq). In certain embodiments, the method for generating the genome-wide transcription profile is selected based on its ability to detect short, unstable eRNAs. In a particular embodiment, the GRO-seq protocol is used to generate the genome-wide nascent transcription profile.

In some embodiments, existing genome-wide nascent transcription profile datasets may be used to predict TF activity in a cell. This may obviate an end user's need to generate a genome-wide nascent transcription profile themselves. The Gene Expression Omnibus (GEO) database, maintained by the National Center for Biotechnology Information (NCBI), is a public functional genomic data repository, and is one source for existing genome-wide nascent transcription profiles. Datasets representing different cell types, disease states, growth conditions, and experimental conditions are available, thus allowing the prediction of TF activity in certain cell types, diseases, or in a cell type treated with a particular drug base on existing data. Generation of new data sets may be necessary, however, to examine TF activity in cells, diseases, or with drugs for which there is no existing dataset.

In certain embodiments, eRNA origination sites are identified in a cell's genomic DNA. The eRNA origination sites may be identified by analyzing a genome-wide nascent transcription profile for the cell. This analysis can be done by several different methods, including but not limited the Transcription fit (Tfit) method (Azofeifa and Dowell, Bioinformatics, (2017) 33(2):227-34, the disclosure of which is hereby incorporated by reference in its entirety), the dREG method (Dank et al., Nat. Meth., (2015) 12(5):433-38, the disclosure of which is hereby incorporated by reference in its entirety), the groHMM method (Chae et al., BMC Bioinformatics, (2015) 16:222, the disclosure of which is hereby incorporated by reference in its entirety), the Vespucci method (Allison et al., Nucleic Acids Res, (2013) 42(4):2433-47, the disclosure of which is hereby incorporated by reference in its entirety), and the FStitch method (Azofeifa et al., Proceeding of the 5th ACM Conference on Bioinformatics, Computational Biology, and Health Informatics, (2014) pp. 174-183, the disclosure of which is hereby incorporated by reference in its entirety). Tfit leverages the known behavior of polymerase II to identify individual transcripts within nascent transcription data. Whether bidirectional (2 transcripts) or unidirectional (1 transcript), the Tfit model precisely infers the point of RNA polymerase lading, e.g., the origin point of transcription. The Tfit method is capable of estimating sites of bidirectional transcript initiation at single base-pair resolution. In some embodiments, identification of eRNA origination sites in the cell's genomic DNA is done by analyzing a genome-wide nascent transcription profile for a cell using the Tfit method (Azofeifa and Dowell, 2017).

In some embodiments, TF DNA binding motif instances for each TF to be studied are identified in the cell's genomic DNA. This can be done for all TFs in a cell (or at least all known TFs, or those for which DNA binding models are known; i.e., on a global scale), for a subset of TFs, or a single TF. A prediction of TF activity can be made for those TFs whose DNA binding motif models have been identified. There are approximately 1,400 TFs encoded within the human genome. Chromatin immunoprecipitation (ChIP-seq) studies have identified binding sites form many of these TFs, allowing examination of a consensus DNA binding motif for more than 600 TFs. Additional databases describing TF binding motif models are also available (e.g., HOCOMOCO; Kulakovskiy et al., Nucleic Acids Research, (2013) 41, D195-D2023, JASPAR CORE databases available at jaspar.binfku.dk, and footprintDB, which includes several other transcription factor binding motif databases, available at floresta.eead.csic.es/footprintdb/?databases). Methods for scanning for TF DNA binding motifs are well known in the art. Representative algorithms for performing such a scan include the algorithm outlined by Staden (Staden: Searching for Motifs in Nucleic Acid Sequences, 93-102 (Springer New York, Totowa, N.J., 1994)) and the MEME suit of motif-based sequence analysis tools (Bailey et al., Nucleic Acids Research, (2009), 37:W202-W208).

In some embodiments, a distance in base pairs is then measured for each identified TF DNA binding motif to at least one of the eRNA origination sites. In certain embodiments, the distance from a DNA binding motif is measured to the nearest eRNA origination site. In other embodiments, the distance from a DNA binding motif is measured to any eRNA origination site within 3,000 bp (3 kb). In yet other embodiments, the distance from a DNA binding motif is measured to any eRNA origination site within 1,500 bp.

In some embodiments, a number of DNA binding motif instances for each unique TF occurring within an first radius (h) of each of the eRNA origination sites (i.e., within h on either side of each eRNA origination site) and the number of number of TF DNA binding motif instances for each unique TF occurring within a second radius (H) of each of the eRNA origination sites (i.e., within H on either side of each eRNA origination site) is determined. In certain embodiments, the h-radius and the H-radius are each centered at each of the eRNA origins and the H-radius is greater than the h-radius. In some embodiments, the h-radius is from 50 bp to 200 bp and the H-radius is from 500 bp to 3,000 bp. In some embodiments, the H-radius is 7-13 times greater than the h-radius. In some embodiments, the H-radius is 10 times greater than the h-radius. In certain embodiments, the h-radius is 150 bp and the H-radius is 1,500 bp.

In certain embodiments, an observed motif-displacement level (MD-level) is calculated for a given TF based on the number of DNA binding motif instances for that transcription factor occurring within the first radius (h) of the eRNA origination sites and the number of DNA binding motif instances for that one transcription factor occurring within the second radius (H) of the eRNA origination sites. In some embodiments, the observed MD-level is calculated by dividing the number of DNA binding motif instances for that TF occurring with the h-radius by the number of DNA binding motif instances for that TF occurring within the H-radius. In certain embodiments, an MD-level is calculated for each TF for which at least one DNA binding motif was identified within an H-radius of an eRNA origination site. Thus, many MD-level can be calculated, each representative of a single TF.

The observed MD-level relates the proportion of significant motif sites within some window 2*h (the h-radius) divided by the total number of motifs against some larger window 2*H (the H-radius) centered at all bidirectional origin sites (eRNA origin sites). It is calculated on a per-position weight matrix (PWM) binding model basis. In certain embodiments, Xⱼ={x₁, x₂, . . . } is a set of bidirectional origin locations genome wide for some experiment j, Yᵢ={y₁y₂, . . . } is a set of all significant motif sites for some TF-DNA binding motif model i genome wide, and the MD-level is calculated according to equation 10:

$$g ( {X}_{j} , {Y}_{i} ; a ) = {\sum }_{x ∈ {X}_{j}} {\sum }_{y ∈ {Y}_{i}} δ ( \vert x - y \vert < a ) \\ {md}_{j , i} = g ( {X}_{j} , {Y}_{i} ; h ) / g ( {X}_{j} , {Y}_{i} ; H ) \\ {md}_{j , i} ∈ [ 0 , 1 ) if h < H . \qquad ( equation 10 )$$

Here, δ(⋅) is an indicator function that returns one if the condition (⋅) evaluates true otherwise to zero. The double sum, i.e. g(a), is naively O(|X∥Y|) however data structures like interval trees reduce time to O(|X|log|Y|).

In some embodiments, an expected MD-level is calculated for each TF, as described in the Materials and Methods section MD-level Significance Under a Non-Stationary Background Model.

In certain embodiments, the observed MD-level is compared to the expected MD-level, and TF activity is predicted if the observed, or calculated MD-level is greater than the expected MD-level. In certain embodiments, TF activity is predicted if the difference between the observed MD-level and the expected MD-level is biologically significant. In some embodiments, the difference between observed MD-level and expected MD-level is biologically significant if p<10⁻³. In other embodiments, the difference is biologically significant if p is less than 10⁻⁴, 10⁻⁵, or 10⁻⁶. In certain embodiments, the difference is biologically significant if p is less that 10′.

In other aspects, embodiments provide methods for evaluating altered transcription factor activity in a cell. The methods are similar to those described above, but rather than comparing an observed MD-level to an expected MD-level, MD-levels for each TF are determined before and after a stimulus is applied to the cell. This allows for approximating the effects of the stimulus on the TF activity in the cell. In some embodiments, the methods allow for the determination of whether the applied stimulus alters TF activity. In certain embodiments, a stimulus may be, for example, a small molecule drug, a biologic, a compound or combination of compounds capable of initiating cellular differentiation or causing a disease state, environmental stressors, time, or any combination of these. In certain embodiments, methods for predicting altered TF activity in a cell included calculating a first MD-level for each TF as described above prior to the application of a stimulus, applying a stimulus to the cell, and calculating a second MD-level for each TF. In some embodiments, a change in transcription factor activity is found to have been caused by the stimulus if the difference between the second MD-level (after stimulus) and the first MD-level (before stimulus) is biologically significant. The activity of a TF is approximated (i.e., inferred) as increased by the stimulus if the second MD-level for that TF is greater than the first MD-level, approximated as decreased if the second MD-level for that TF is smaller than the first MD-level, or approximated as unchanged if the second MD-level for that TF is approximately equal to the first MD-level. Where two observed MD-levels are compared, biological significance may be determined as described in the Materials and Methods section MD-level Significance Between Experiments, where p is significant if less than one of 10⁻³, 10⁻⁴, 10⁻⁵, or 10⁻⁶. In certain embodiments, the difference is biologically significant if p is less that 10⁻⁶.

In certain embodiments, existing datasets representing genome-wide nascent transcript profiles for a same cell type can be used to determine alterations in TF activity, where the dataset provides a transcript profile for the same cell type before and after treatment with some stimulus. Examples of identifying alterations in TF activity are provided in Example 1, where pairwise comparisons are made between cells treated with Nutlin-3a, TNFα, or estradiol, each of which are known to affect transcription factor activity. In such embodiments, it will be recognized that the stimulus is not applied by the user. However, an observed MD-level is still determined for a cell type both before and after application of a stimulus.

In other embodiments, a user may generate its own genome-wide nascent transcript profile before application of a stimulus, after application of a stimulus to a cell, or both. For example, to investigate the effect of a drug on a particular cell type for which there exists a genome-wide nascent transcript file under control conditions but not following application of the drug of interest, the method for predicting altered transcription may include determining the first MD-level from the existing data set, applying a stimulus to pair-matched cells, generating a new post-stimulus genome wide nascent transcript profile, and calculating a post-stimulus MD-level.

It will be recognized that the stimulus is not necessarily applied to the same individual cell or group of cells used to generate the pre-stimulus transcript profile, but rather to the same cell type, to allow pairwise comparison between pre- and post-stimulus MD-levels.

The methods described herein may be used to approximate TF activity or alterations in TF activity for any cell type, whether originating from human, animal, plant, or microorganism. The only requirements for use of the present methods are that the cell type be amenable to genome-wide nascent transcript sequencing and that at least a subset of TF binding motifs be available for the cell type of interest.

Certain embodiments provide methods for ascertaining a set of prospectively active transcription factors. In some embodiments, methods of the present disclosure can be used to ascertain a set of transcription factors predicted to be active in a given wild-type cell, in a diseased cell, or in a cell following a perturbation. In some embodiments, a method can further include confirming activity of each of the transcription factors of the ascertained set of transcription factors. In certain embodiments, this can be done evaluating transcription factor binding to the cell's DNA. In some embodiments, techniques useful for evaluating transcription factor binding to the cell's DNA include, but are not limited to, one or more of ChIP-seq, ATAC-seq, DNAas-seq, and FAIRE-seq.

In some embodiments, ascertaining a set of prospectively active transcription factors according to methods of the disclosure can significantly reduce the time and cost required to identify and confirm transcription factor involvement in, for example, a particular cell type, cell process, disease progression, and a cell's response to a perturbation. By first ascertaining a set of prospectively active transportation factors, it is possible to target further studies to those identified transcription factors, eliminating the need for a “shotgun” approach and individually evaluating a broad range of transcription factors.

In certain embodiments ascertaining a set of prospectively active transcription factors according to methods of the disclosure can provide guidance as to which transcription factors may be cell-type specific (e.g., markers of cell type), and may be targeted in order to effect cellular reprogramming.

In some embodiments, ascertaining a set of prospectively active transcription factors according to methods of the disclosure can provide guidance as to which transcription factors may be targeted for drug development. Many FDA-approved drugs modulate TF activity, such as BPA (modulates ESRRG), dihydrotestosterone (ANDR), decitabine (DNMT1), T-5224 (AP-1), TNF-α (NF-κB1), thiazolidinedione (PPARγ), tamibarotene (RARα), calcitrol (VDR), and nutlin-3a (TP53). In some embodiments, the methods provided herein can identify transcription factors active in a disease state. In other embodiments, the methods described can identify those transcription factors affected by a particular perturbation, including administration of a small molecule or a biologic. Identifying a set of transcription factors according to the embodiments of the disclosure can increase overall drug screening throughput capabilities by targeting further studies to a limited set of transcription factors. Further, the disclosed methods can identify a small set of prospectively active transcription factors affected by a given compound, thereby identifying a likely drug target and enabling drug screens for other compounds capable of affecting activity of one or more transcription factors of the small set of identified transcription factors.

Given the massive amounts of data required by the present methods, in particular embodiments, the methods provided herein may be implemented by a computer system. In certain embodiments, a processor of a computer system accesses a database that includes a genome-wide nascent transcription profile for a cell, and carries out the steps described above, including identifying eRNA origination sites and DNA binding motif instances, calculating an observed MD-level and an expected MD-level, and predicting the TF activity in the cell. Other embodiments provide a computer implemented method for predicting altered transcription factor activity in a cell, including the steps of accessing a database that includes genome-wide nascent transcription profiles for a cell before and after a stimulus has been applied to the cell and calculating a first pre-stimulus MD-level and a second post-stimulus MD-level, and predicting alterations in TF activity.

Referring now to FIG. 19, several embodiments of the present disclosure (as well as environments in which they operate) can utilize one or more computers or other processor-based laboratory systems (250) connected over a network 262, such as the Internet. The illustrated processor-based system 250 includes a processor 254 coupled to a memory 256 and a network interface 258 through a bus 260. The network interface 258 is also coupled to a network 262 such as the Internet. In other embodiments, the processor-based system 250 may include a plurality of components (e.g., a plurality of memories 256 or buses 260). The network 262 may include a remote data storage system including a plurality of remote storage units 264 configured to store data at remote locations. Each remote storage unit 264 may be network addressable storage. In some embodiments, the processor-based system 250 includes a computer-readable medium containing instructions that cause the processor 254 to perform specific functions described herein. That medium may include a hard drive, a disk, memory, or a transmission, among other computer-readable media. In some embodiments, the processor-based system 250 is integrated into a genetic sequencer. In other embodiments, the processor-based system 250 in connected to a genetic sequencer (e.g., via network 262). In yet other embodiments, genetic sequencing information is stored in a database (e.g., database 264) accessed by the processor-based system 250.

Certain embodiments provide a laboratory method for evaluating the effect of a stimulus on a cell using a Motif-Displacement model (MD) that approximates transcription factor activity in the cell. In some embodiments, the laboratory method includes i) locating a first set of enhancer RNA (eRNA) origination sites in the cell's genomic DNA using a first genome-wide nascent transcription profile for the cell, ii) identifying DNA binding motif instances for transcription factors in the cell's genomic DNA, iii) for each eRNA origination site in the first set of eRNA origination sites, measuring a number of DNA binding motif instances for each of the transcription factors occurring within a first radius of the eRNA origination site and measuring a number of DNA binding motif instances for each of the transcription factors occurring within a second radius of the eRNA site, wherein the first radius and the second radius are each centered at each of the eRNA origination sites of the first set of eRNA origination sites, and the second radius is greater than the first radius, iv) calculating a first MD-level for each of the transcription factors based on the number of DNA binding motif instances for that transcription factor occurring within the first radius of the eRNA origination sites of the first set of eRNA origination sites and the number of DNA binding motif instances for that one transcription factors occurring within the second radius of the eRNA origination sites of the first set of eRNA origination sites, v) applying a stimulus to the cell, vi) locating a second set of eRNA origination sites in the cell's genomic DNA using a second genome-wide nascent transcription profile for the cell, wherein the second genome-wide nascent transcription profile is generated after applying the stimulus to the cell, vii) for each eRNA origination site in the second set of eRNA origination sites, measuring a number of DNA binding motif instances for each of the transcription factors occurring within the first radius of the eRNA origination site and measuring a number of DNA binding motif instances for each of the transcription factors occurring within the second radius of the eRNA site, viii) calculating a second MD-level for each of the transcription factors based on the number of DNA binding motif instances for that transcription factor occurring within the first radius of the eRNA origination sites of the second set of eRNA and the number of DNA binding motif instances for that one transcription factors occurring within the second radius of the eRNA origination sites of the second set of eRNA origination sites, and ix) approximating effects of the stimulus on the transcription factor activity in the cell by identifying biologically significant differences between the first MD-level and the second MD-level.

In some embodiments, the laboratory method is a processor-based laboratory method, wherein at least some of the steps are performed by one or more processors executing instructions stored in a tangible, non-transitory storage medium. In some embodiment, all steps of the processor-based laboratory method are performed by one or more processors executing instructions stored in a tangible, non-transitory storage medium. In some embodiments, at least the calculating steps are performed by one or more processors executing instructions stored in a tangible, non-transitory storage medium.

In some embodiments, locating the sets of eRNAs is performed as described above. In certain embodiments, locating the sets of eRNAs are performed by one or more processors executing instructions stored in a tangible, non-transitory storage medium, wherein the one or more processors execute instructions according to the Tfit method, the dREG method, the groHMM method, the Vespucci method, or the FStitch method.

In some embodiments, identifying DNA binding motif instances for transcription factors in the cell's genomic DNA is carried out as described above, wherein the identifying is carried out by the one or more processors.

In some embodiments, the one or more processors executes instructions to measure a number of DNA binding motif instances for each of the transcription factors occurring within a first radius of an eRNA origination site and measure a number of DNA binding motif instances for each of the transcription factors occurring within a second radius of an eRNA origination site.

In some embodiments, the one or more processors executes instructions to approximate the effects of the stimulus on the transcription factor activity in a cell by identifying biologically significant differences between a first MD-level calculated prior to a stimulus and a second MD-level calculated following application of a stimulus. Biological significance can be determined as described above.

Some embodiments provide computer-based systems for evaluating the effect of a stimulus on a cell using a Motif-Displacement model described herein that approximates transcription factor activity in a cell. In some embodiments, the system includes one or more processors and a non-transitory, tangible storage medium containing instructions that, when executed by the processor, can cause the one or more processors to carry out one or more of the disclosed methods. In some embodiments, the instructions cause the one or more processors to i) locate a first set of enhancer RNA (eRNA) origination sites in the cell's genomic DNA using a first genome-wide nascent transcription profile for the cell, ii) identify DNA binding motif instances for transcription factors in the cell's genomic DNA, iii) for each eRNA origination site in the first set of eRNA origination sites, measuring a number of DNA binding motif instances for each of the transcription factors occurring within a first radius of the eRNA origination site and measuring a number of DNA binding motif instances for each of the transcription factors occurring within a second radius of the eRNA site, wherein the first radius and the second radius are each centered at each of the eRNA origination sites of the first set of eRNA origination sites, and the second radius is greater than the first radius, iv) calculate a first MD-level for each of the transcription factors based on the number of DNA binding motif instances for that transcription factor occurring within the first radius of the eRNA origination sites of the first set of eRNA origination sites and the number of DNA binding motif instances for that one transcription factors occurring within the second radius of the eRNA origination sites of the first set of eRNA origination sites, v) locate a second set of eRNA origination sites in the cell's genomic DNA using a second genome-wide transcription profile for the cell, wherein the second genome-wide nascent transcription profile is generated after applying a stimulus to the cell, vi) for each eRNA origination site in the second set of eRNA origination sites, measuring a number of DNA binding motif instances for each of the transcription factors occurring within the first radius of the eRNA origination site and measuring a number of DNA binding motif instances for each of the transcription factors occurring within the second radius of the eRNA site, vii) calculate a second MD-level for each of the transcription factors based on the number of DNA binding motif instances for that transcription factor occurring within the first radius of the eRNA origination sites of the second set of eRNA origination sites and the number of DNA binding motif instances for that one transcription factors occurring within the second radius of the eRNA origination sites of the second set of eRNA origination sites, and viii) approximate effects of the stimulus on the transcription factor activity in the cell by identifying biologically significant differences between the first MD-level and the second MD-level.

Some embodiments provide a processor-based method for identifying active transcription factors in a cell. In some embodiments, the methods include i) locating enhancer RNA (eRNA) origination sites in the cell's genomic DNA by analyzing a genome-wide nascent transcription profile for the cell, ii) identifying DNA binding motif instances for transcription factors in the cell's genomic DNA, iii) measuring a number of DNA binding motif instances for each of the transcription factors occurring within a first radius of each of the eRNA origination sites, iv) measuring a number of DNA binding motif instances for each of the transcription factors occurring within a second radius of each of the eRNA origination sites, wherein the first radius and the second radius are each centered at each of the eRNA origination sites and wherein the second radius is greater than the first radius, v) using one or more processors to determine a Motif-Displacement (MD) level that approximates transcription factor activity in the cell, the processor executing instructions stored in a tangible, non-transitory storage medium in order to: a) calculate an MD-level for each of the transcription factors using the number of DNA binding motif instances for a transcription factor occurring within the first radius of the eRNA origination site and the number of DNA binding motif instances for that transcription factor occurring within the second radius of the eRNA origination site, b) calculate an expected MD-level for each of the at least one transcription factors, and c) allocate the at least one transcription factor as active in the cell if the calculated MD-level is greater than the expected MD-level and if the difference between the calculated MD-level and the expected MD-level is biologically significant. In other embodiments, in addition to step v), one or more of steps i)-iv) can be performed by the one or more processors.

Some embodiments provide a TF prediction system for modelling transcription factor activity in a cell. In certain embodiments, the TF prediction system includes a database have at least one genome-wide nascent transcription profile for a cell, and a TF analyzer communicatively coupled to the database. In certain embodiments, the TF analyzer is configured to carry out the steps of a method described herein, including, for example, analyzing a genome-wide nascent transcription profile to identify eRNA origination sites, identifying DNA binding motifs for at least one TF and measuring the distance from the motif instances to at least one of the eRNA origination sites, calculating an observed MD-level and an expected MD-level for each TF, and predicting the TF activity.

Some embodiments provide TF prediction systems for predicting alterations in transcription factor activity in a cell. In certain embodiments, the TF prediction system includes a database having at least one pair of genome-wide nascent transcription profiles for a cell, and a TF analyzer communicatively coupled to the database. In certain embodiments, the TF analyzer is configured to carry out the steps of a method described herein, including, for example analyzing the pair of genome-wide nascent transcription profiles to identify eRNA origination sites in each profile, analyzing each profile to identify eRNA origination sites, identifying DNA binding motif instances for at least one TF and for each profile measuring the distance from the motif instances to at least one of the eRNA origination sites, calculating an observed MD-level for each TF in each profile, and predicting alterations in TF activity between the two profiles. In certain embodiments, a first profile reflects a cell type's nascent transcripts prior to a stimulus and a second profile reflects a cell type's nascent transcripts after a stimulus.

In come embodiments, the database is communicatively coupled to the TF analyzer by a communication link. In embodiments, the communication link may be, or include, a wired communication link such as for example, a USB link, a proprietary wired protocol, and/or the like. The communication link may be, or include, a wireless communication link such as, for example, a short-range radio link, such as Bluetooth, IEEE 802.11, a proprietary wireless protocol, and/or the like. In embodiments, for example, the communication link may utilize Bluetooth Low Energy radio (Bluetooth 4.1), or a similar protocol, and may utilize an operating frequency in the range of 2.40 to 2.48 GHz.

The term “communication link” may refer to an ability to communicate some type of information in at least one direction between at least two devices, and should not be understood to be limited to a direct, persistent, or otherwise limited communication channel That is, according to embodiments, the communication link may be a persistent communication link, an intermittent communication link, an ad-hoc communication link, and/or the like. The communication link may refer to direct communications between the database and the TF analyzer, and/or indirect communications that travel between the database and the TF analyzer via at least one other device (e.g., a repeater, router, hub, and/or the like). The communication link may facilitate uni-directional and/or bi-directional communication between the database and the TF analyzer. In embodiments, the communication link is, includes, or is included in a wired network, a wireless network, or a combination of wired and wireless networks. Illustrative networks include any number of different types of communication networks such as, a short messaging service (SMS), a local area network (LAN), a wireless LAN (WLAN), a wide area network (WAN), the Internet, a peer-to-peer (P2P) network, or other suitable networks. The network may include a combination of multiple networks. In embodiments, for example, the TF analyzer may be accessible via the Internet (e.g., the TF analyzer may facilitate a web-based TF analysis service), and a user may transmit one or more genome-wide nascent transcript profiles to the TF analyzer to predict TF activity.

In some embodiments, the TF analyzer accesses the database via the communication link. The database may be web-based, cloud based, or local. In embodiments, the database is retrieved from a third party, produced by the user, or some combination thereof. The database may be any collection of information providing one or more nascent transcript profiles.

In some embodiments, the TF analyzer is implemented on a computing device that includes a processor, a memory, and an input/output (I/O) device. Although the TF analyzer is referred to herein in the singular, the TF analyzer may be implemented in multiple instances, distributed across multiple computing devices, instantiated within multiple virtual machines, and the like. In embodiments, the processor executes various program components stored in the memory, which may facilitate predicting TF activity. In embodiments, the processor may be, or include, one processor or multiple processors. In embodiments, the I/O component may be, or include, one I/O component or multiple I/O components and may be, or include, any number of different types of devices such as, for example, a monitor, a keyboard, a printer, a disk drive, a universal serial bus (USB) port, a speaker, pointer device, a trackball, a button, a switch, a touch screen, and/or the like. Alternatively, or additionally, the I/O component may include software and/or firmware and may include a communication component configured to facilitate communication via the communication link, and/or the like.

According to embodiments, as indicated above, various components of the TF prediction system may be implemented on one or more computing devices. A computing device may include any type of computing device suitable for implementing embodiments of the invention. Examples of computing devices include specialized computing devices or general-purpose computing devices such as “workstations,” “servers,” “laptops,” “desktops,” “tablet computers,” “hand-held devices,” and the like, all of which are contemplated within the scope the TF prediction system. For example, according to embodiments, the TF analyzer may be, or include, a general purpose computing device (e.g., a desktop computer, a laptop, a mobile device, and/or the like), a specially-designed computing device (e.g., a dedicated device), and/or the like.

In some embodiments, a computing device includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof) Similarly, in embodiments, the computing device may include a number of processors, a number of memory components, a number of I/O ports, a number of I/O components, and/or a number of power supplies. Additionally any number of these components, or combinations thereof, may be distributed and/or duplicated across a number of computing devices.

In some embodiments, memory includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; or any other medium that can be used to store information and can be accessed by a computing device such as, for example, quantum state memory, and the like. In embodiments, the memory stores computer-executable instructions for causing the processor to implement aspects of embodiments of system components discussed herein and/or to perform aspects of embodiments of methods and procedures discussed herein. Computer-executable instructions may include, for example, computer code, machine-useable instructions, and the like such as, for example, program components capable of being executed by one or more processors associated with a computing device. Examples of such program components include an eRNA identifying component, a TF DNA binding motif component, an MD-level calculating component, and a TF activity prediction component. Program components may be programmed using any number of different programming environments, including various languages, development kits, frameworks, and/or the like. Some or all of the functionality contemplated herein may also, or alternatively, be implemented in hardware and/or firmware.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Example 1—Enhancer RNA Profiling Predicts Transcription Factor Activity

Transcription factors (TFs) exert their regulatory influence through the binding of enhancers, resulting in coordination of gene expression programs. Active enhancers are often characterized by the presence of short, unstable transcripts termed enhancer RNAs (eRNAs). While their function remains unclear, the studies presented herein demonstrate that eRNAs are a powerful readout of TF activity. Sites of eRNA origination were inferred across hundreds of publicly available nascent transcription datasets. eRNAs were demonstrated to initiate from sites of TF binding. By quantifying the co-localization of TF binding motif instances and eRNA origins, a statistic capable of inferring TF activity, termed Motif-Displacement score (MD-level). In doing so, dozens of previously unexplored links between diverse stimuli and the TFs they affect were uncovered. The approach described herein constitutes a fundamentally unique strategy for predicting TF activity, identifying alterations in TF activity following cellular perturbation (e.g., by drugs) or perturbation, and discovering links between TF activity and phenotypes.

Enhancer RNAs Originate from Transcription Factor Binding Sites

eRNA detection requires extremely sensitive methods, both in the laboratory as well as computationally. Because they are unstable, eRNAs are hard to capture via steady state RNA assays such as RNA-seq. Nascent transcription assays capture transcription throughout the genome, including eRNA transcription. A model (Tfit) capable of estimating sites of bidirectional transcript initiation at single base-pair resolution was recently described by the inventors (Azofeifa and Dowell, 2017, Bioinformatics, 33(2):227-234, the disclosure of which is incorporated herein by reference in its entirety). This model was used to generate sets of eRNA origin sites.

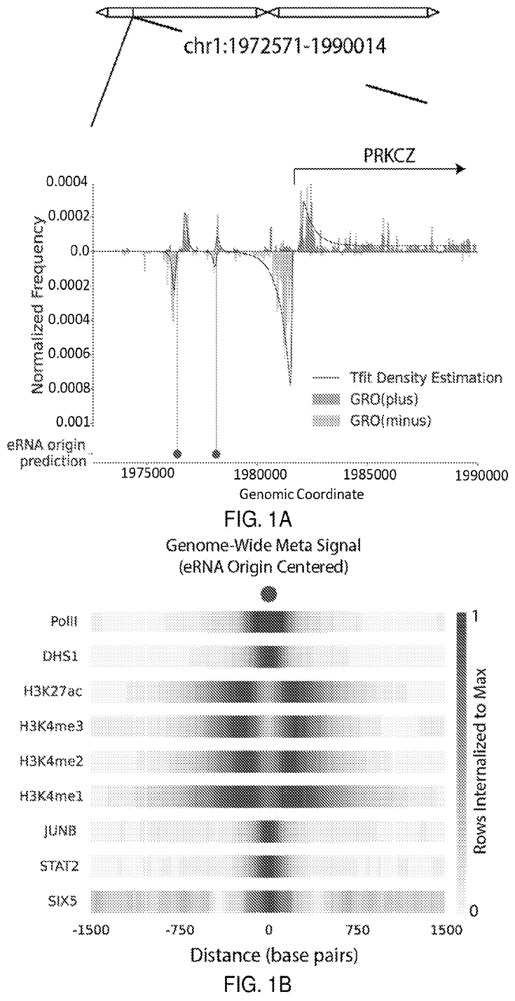

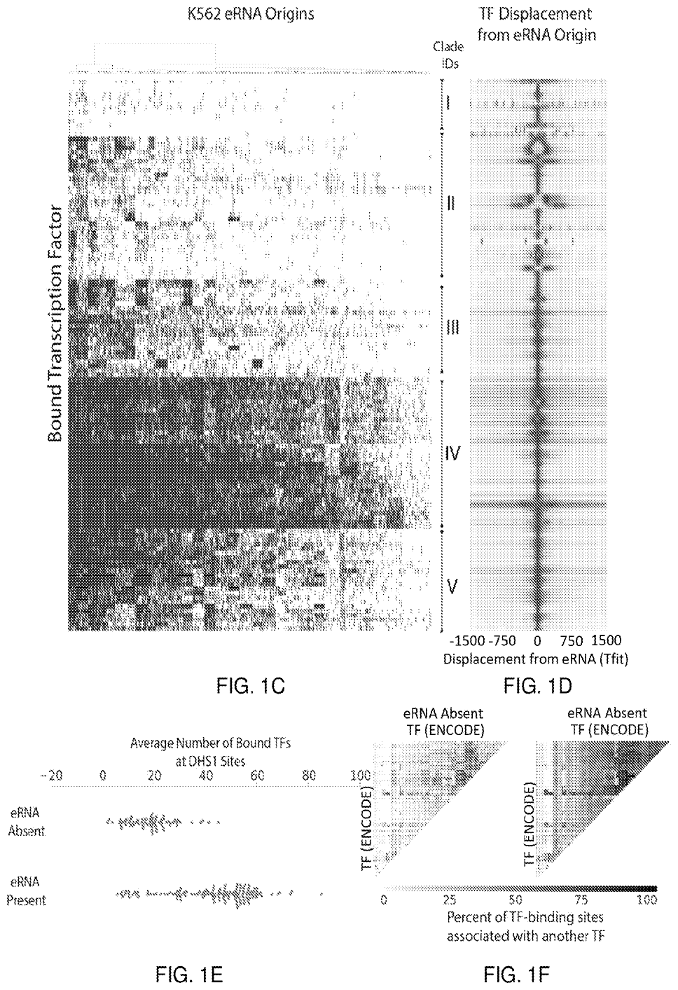

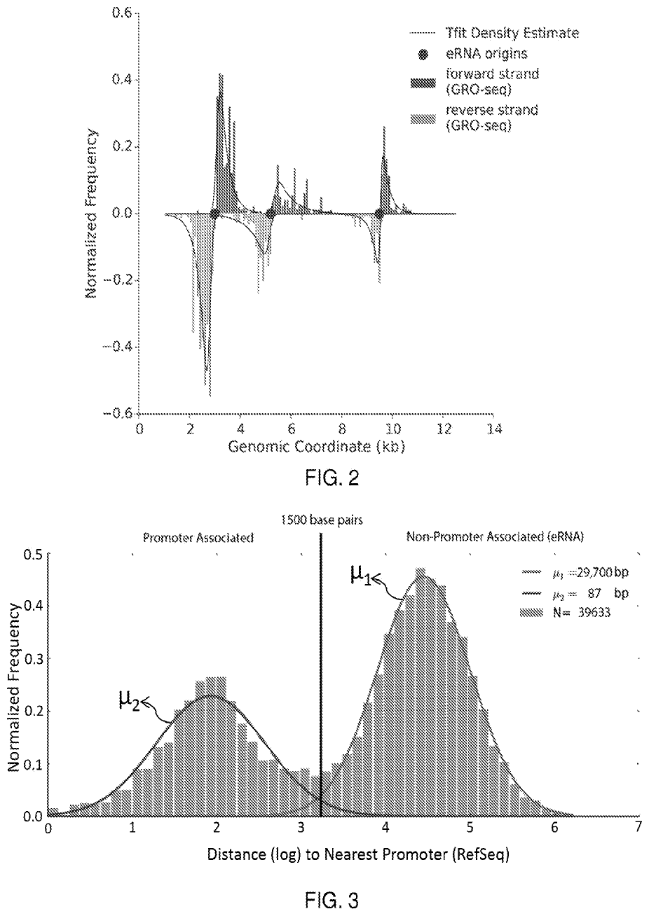

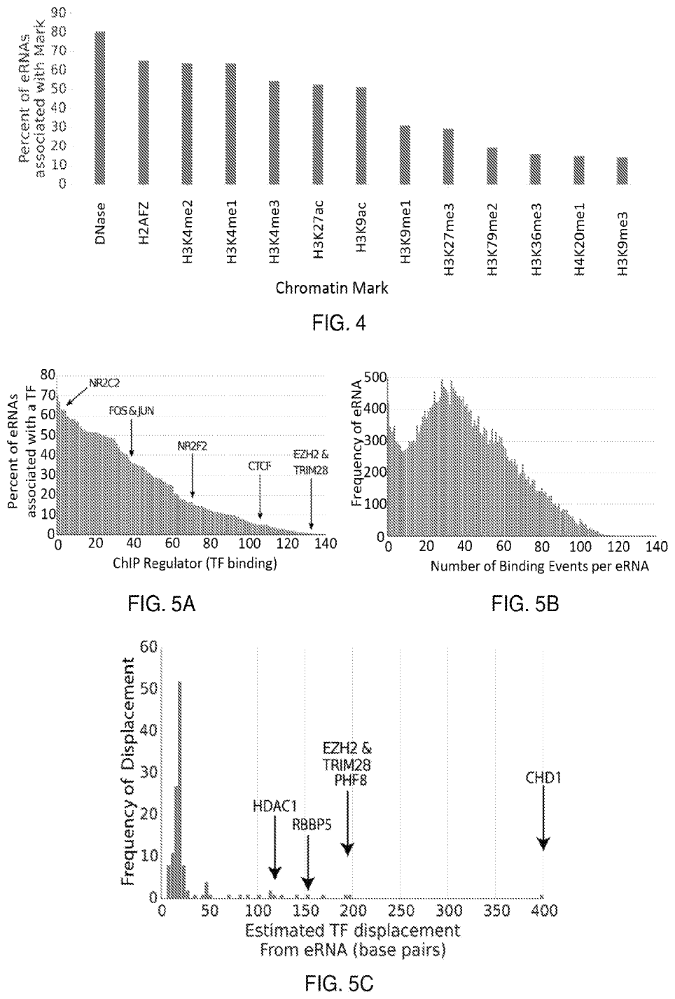

In order to profile enhancer transcription genome-wide, 39,633 putative sites of bidirectional transcription in a K562 GRO-cap dataset were identified, of which 30,324 were not associated with an annotated promoter (FIG. 1A, FIG. 2, and FIG. 3). As previously observed (Azofeifa and Dowell, 2017), DNase I hypersensitivity (DHS), histone 3 lysine 27 acetylation (H3K27ac), and histone 3 lysine 4 mono-, di-& tri-methylation significantly associate with non-promoter bidirectional transcription (FIG. 1B and FIG. 4). Histone modifications are displaced from bidirectional centers, supporting the presence of a nucleosome-free region localized precisely at the origins of bidirectional transcript initiation (FIG. 1B). Given their overwhelming co-occurrence with marks of active and open chromatin, as well as their distal location relative to annotated promoters, these transcripts are referred to herein as enhancer RNAs (eRNAs).

FIG. 1A depicts an exemplary locus displaying nascent transcript sequencing read coverage (HCT116 GRO-seq) with the overlaid density estimation via Tfit and the associated eRNA origin predictions (dots). FIG. 1B represents a genome wide meta-signal for marks of active chromatin aligned to eRNA origins inferred by Tfit in a K562 GRO-cap dataset (Core et al., 2014, Nat Genet 46:1311-20).

FIG. 2 displays an annotated super enhancer (starting at chr2:10, 456,371) indicating the final inferred density function obtained by Tfit (Azofeifa and Dowell, 2017). Via Bayesian model selection, three distinct eRNA origins are identified. Green dots indicate the eRNA origin estimate.

As indicated by FIG. 3, most sites of bidirectional transcription identified by Tfit lack association with promoter regions. Sites of bidirectional transcription were profiled in a K562 GRO-cap (SRR1552480) experiment using Tfit (see Tfit Parameters and Bidirectional Prediction). A promoter is defined as the region associated with a RefSeq (release 76) annotated gene's start site. Bimodiality was estimated via a two component Gaussian mixture model fit with the EM algorithm. The mean of each Gaussian curve is given in the key.

The bar graph presented in FIG. 4 indicates that sites of non-promoter associated bidirectional transcription overlap marks of regulatory DNA. The percentage of eRNAs in a K562 GRO-cap data set (SRR1552480) that associate with specific chromatin marks, defined by ENCODE. Promoter-associated Tfit predictions were removed from this analysis.

To assess eRNA and TF binding co-occurrence in a more systematic fashion, a generated set of eRNA origins was integrated with the genomic binding locations of 139 proteins profiled by ChIP-seq, also in K562 cells. 98% of eRNAs are bound by at least one regulator, where an average of 52.9 regulators localize at any one eRNA (FIGS. 5A-5B). In fact, three distinct patterns of TF binding were observed (FIG. 1C): 1) TFs that bind all eRNAs (32 factors co-occur with over 75% of all eRNAs; clade IV)); 2) TFs that bind only a few eRNAs (39 factors associate with no more than 20% of all eRNAs; clades I & II); and 3) TFs that bind to many eRNAs but only with unique TF partners (58 factors occur under specific combinatorial patterning, e.g. GATA2/NR2F2/GABPA and FOSL/ATF3 strongly co-localize at eRNAs; clades III & V). In summary, unique sets of TFs bind to specific eRNA origins.

FIG. 5A indicates the proportion (y-axis) of a TT's ChIP peaks associated <1.5 kb with an eRNA origin. The x-axis is one of the 129 TFs profiled by ENCODE in K562 cells. FIG. 5B indicates the number of unique TF binding peaks occurring at individual eRNAs.

FIG. 1C represents the overlap of eRNA origins (columns) with 139 regulatory proteins (rows). A tick indicates the presence of TF binding site within 1.5 KB of the Tfit inferred origin; sorted by hierarchical clustering.

For the set of eRNA origins that overlap TF binding sites, the co-localization of TFs relative to eRNA origins was examined (FIG. 1D). Two classes of regulators were observed; 84% of TFs exhibit centered, unimodal localization with eRNA origins and 16% display significantly displaced peak localization flanking eRNA origins (see Computation of Bimodality). For example, factors such as RBB5, PHF8 and CDH1 are significantly displaced an average of 150, 200, and 398 base pairs away from the eRNA origin, respectively (FIG. 5C). Regulators with displaced peak localization are significantly enriched for ontological definitions such as “histone modification,” “chromatin organization,” and “histone deacetylation” consistent with the bimodal distribution of histone modifications observed in FIG. 1B (p-value<10⁻⁶).

FIG. 1D presents a histogram of the spatial displacement of the TF binding peak from eRNA origins (heat is normalized to min/max as in FIG. 1B).

TF displacement data was calculated within a 5 kb radius around eRNA locations and bimodal model selection was performed via a Laplace-Uniform mixture (see Computation of Bimodality, ΔBIC). A larger ΔBIC value indicates greater support for bimodal TF peak displacement. FIG. 5C presents the distribution of estimated peak displacements. All data presented in FIGS. 5A-5C is from K562 cells, both nascent transcription (SRR1552480) and ENCODE ChIP-seq peaks.

In addition to chromatin state, TF-combinatorial control also plays a pivotal role in downstream gene regulation. In general, the number of TFs co-localized at sites of open chromatin is larger when an eRNA is present than not (FIG. 1E). Furthermore, TF co-association dramatically increases when considering eRNA presence (FIG. 1F). Taken together, the localization of diverse binding complexes at eRNA-associated TF binding sites indicates that eRNAs are likely markers of functional transcription factor binding.

FIG. 1E presents a swarm plot displaying the number of bound TFs at sites of open chromatin grouped by eRNA association, while FIG. 1F provides a pairwise co-association map where increased heat indicates a greater proportion of TFs binding sites also bound by another TF; categorized eRNA association.

### Enhancer RNA Origins Mark Sites of Regulatory TF Binding

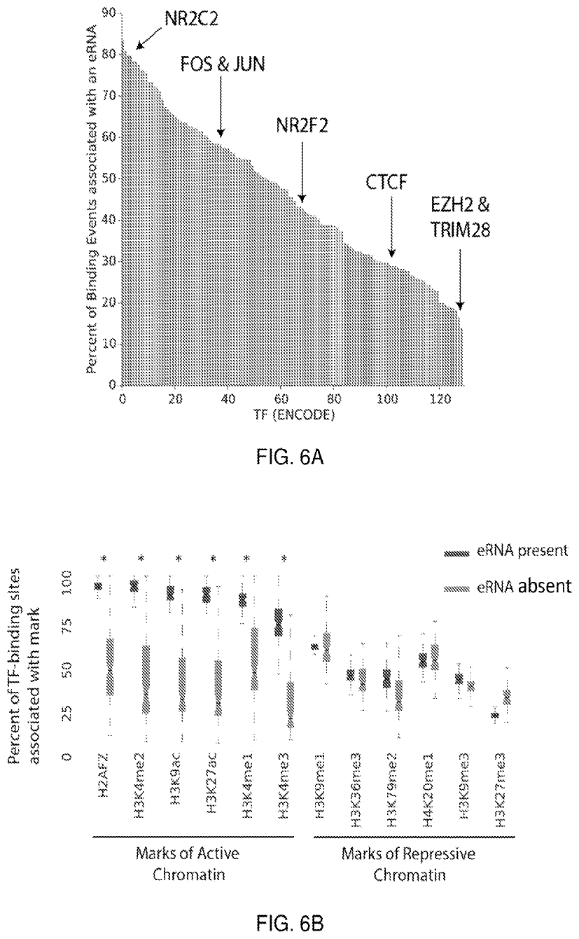

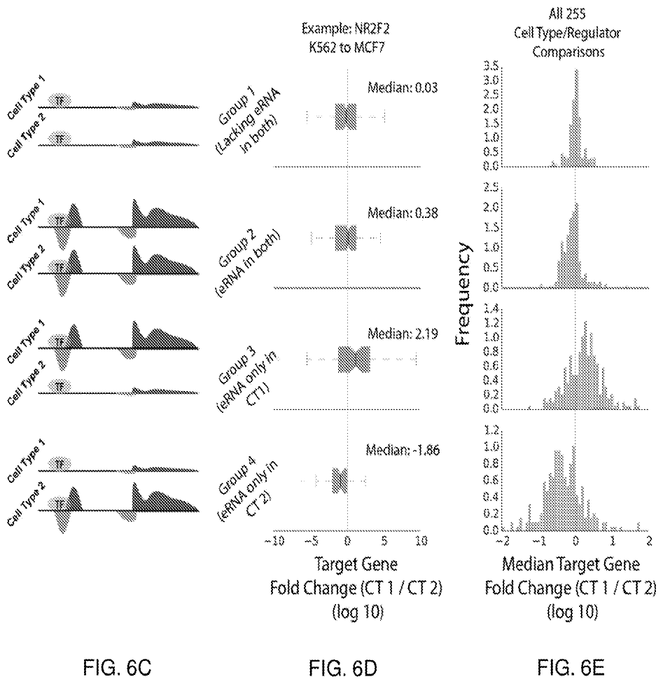

Although the vast majority of eRNA origins localize with TF binding, only a fraction of TF binding sites overlap eRNA origins (FIG. 6A). Previous efforts to predict sites of TF binding using joint eRNA and TF-DNA motifs focused on only a small set of transcription factors. Here, the analysis is extended to include 139 transcription factor ChIP-seq experiments in which a wide spectrum of association between TF binding sites and eRNA presence was observed, indicating that eRNA presence alone is not sufficient to fully explain TF binding. This data is consistent with the observation that only a fraction of TF binding sites result in a concomitant change in nearby gene expression. Given the strong relationship between active chromatin and eRNA transcription, whether eRNAs discriminate “silent” from “active” TF binding was investigated. In support of this hypothesis, TF binding sites occurring at sites of eRNA origination display a significantly increased overlap with canonical marks of active chromatin relative to non-eRNA associated TF binding (FIG. 6B). Moreover, no statistical difference is detected between these categories for repressive chromatin marks.

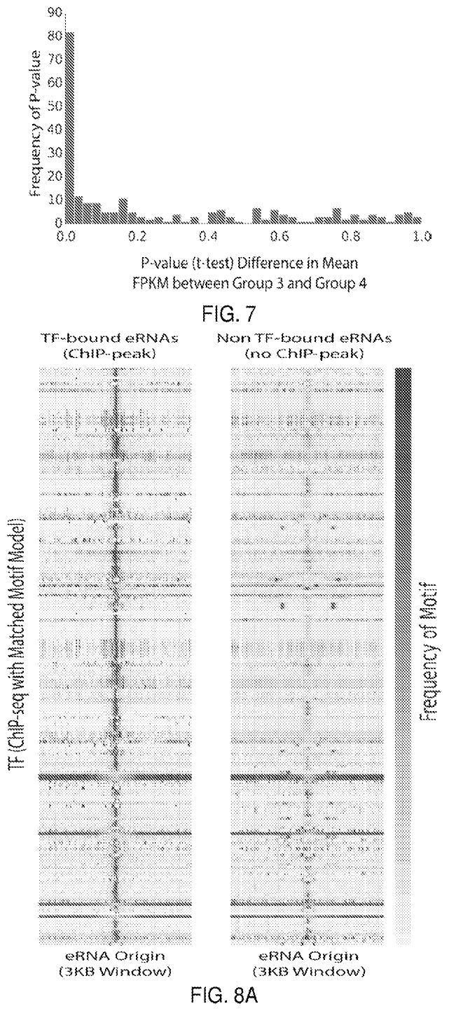

Although regulatory TF binding is often enriched for open and active chromatin, functional TF binding must ultimately lead to a change in gene expression. To this end, TF binding events within enhancers were considered conserved between two cell types but differing in terms of eRNA presence with the hypothesis that neighboring gene expression would be elevated in the eRNA-harboring cell type (FIG. 6C). There are 95 TFs profiled in at least two cell types for which cell type-matched nascent transcription is available. For example, binding of the transcription factor NR2F2 was profiled in both K562 and MCF7 cell lines, yielding 30,618 and 16,678 binding peaks respectively, with 3,491 peaks shared between the two cell types (FIG. 6D). Of these cell type invariant peaks, 25% harbor an eRNA origin in both cell types, 7% only in K562, 12% only in MCF7 and 56% do not harbor an eRNA origin in either cell type. Measuring the transcription level of nearby target genes (TF binding site <10 kb of gene promoter) revealed that eRNA presence is significantly correlated with elevated local gene expression (p-value<10⁻⁶). After making a total of 262 possible pairwise cell type comparisons (95 TF, 4 cell types), it was noted that 73% of these comparisons display such dynamics (FIG. 6E, FIG. 7).

FIG. 6A is a histogram indicating that eRNA presence marks the active subset of TF binding. The y-axis indicates the proportion of a TFs ChIP peaks associated <1.5 KB with an eRNA origin. The x-axis is one of the 129 TFs profiled by ENCODE in K562 cells. FIG. 6B is a box- and -whiskers plot displaying the median/variability in proportion of histone mark association between the groups across all TFs. TF binding peaks were grouped according to eRNA association. Asterisks indicate a p-value<10-10 by z-test. All data in A-B are from K562 cells. FIGS. 6C-6E indicate pairwise cell-type associated TF binding peaks grouped according to eRNA presence from matched cell types. A gene was considered “neighboring” by a distance less than 10 kb. FIG. 6D Log base 10 FPKM fold change of “neighboring” genes related to eRNA-grouped NR2F2 binding peaks. FIG. 6E is a histogram of Lob base 10 FPKM fold change of “neighboring” genes for all possible eRNA-grouped TF ChIP-seq data sets (n=255).

TABLE 1
Cell Type Matched Nascent Transcript Datasets.

| Cell Type | Dataset (SRA) |
|---|---|
| HCT116 | SRR1105737 |
| MCF7 | SRR915731 |
| GM12878 | SRR1552482 |
| K562 | SRR1552480 |
| HeLa | SRR1596500 |

FIGS. 6C-6E presentan analysis of the association between the TF binding sites with eRNAs. TF binding sites (by ChIP) conserved between two cell types were identified. These (non-promoter associated) genomic loci were further categorized as associated with an eRNA in cell type 1 (CT1) and lacking an eRNA in cell type 2 (CT2) or vice versa. Finally, log₂ fold chance in FPKM of genes near these sites (<10 KB) was collected and a two-tailed t-test was used to assess a difference in means. FIG. 7 is a histogram of p-values following the test of the hypothesis that TF binding sites associated with cell type unique eRNAs modulate local gene expression.

A functional assay was used to determine if eRNA presence marked the active subset of TF binding. TF binding sites that overlap a region with strong enhancer activity—as measured by a CapStarr-seq enhancer assay (Vanhille et al., 2015, Nat Commun, 6:6905)—are five times more likely to associate with eRNAs than regions considered inactive by the enhancer assay (p-value<10⁻¹⁹, hypergeometric). These results are consistent with a model where eRNA presence discriminates silent from functional TF binding.

eRNA Origins Co-Localize with TF binding Motif Instances.

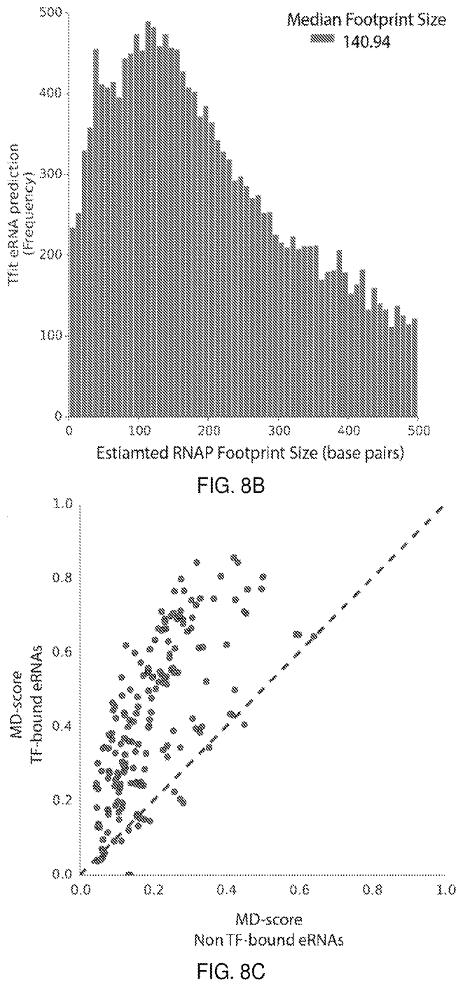

Given that many TFs bind DNA in a sequence specific manner, the precise spatial relationship between instances of the TF DNA motif model and eRNA transcription was determined. To this end, the distance between genomic instances of the TF motif model and eRNA origins in a K562 GRO-cap data set was measured. Co-localization of the motif with the eRNA origin specifically in the TF-bound fraction of eRNAs was observed (FIG. 8A), indicating that the motif sequence is present at the precise point of eRNA origination. This indicated that the genome-wide patterns of motif sequence to eRNA co-occurrence could identify the set of active transcription factors directly regulating eRNA transcription, even when ChIP data is not available.

FIG. 8A provides heatmaps displaying the frequency of TF motif instances centered at the eRNA origin predicted by Tfit from a K562 GRO-cap (SRR1552480) experiment. eRNAs were further separated by association with or distal to a TF by ChIP. Motif models and ChIP-matched data sets gilded 57 unique transcription factors and 187 separate peak files.

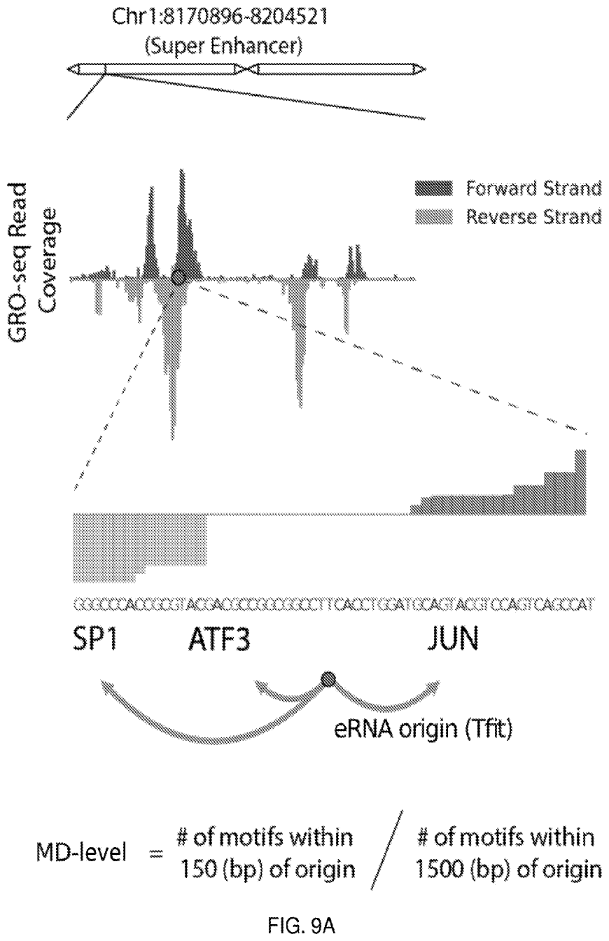

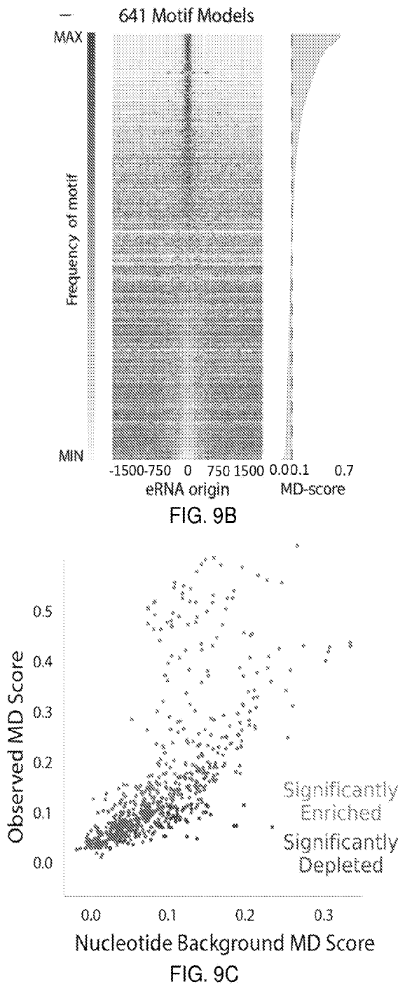

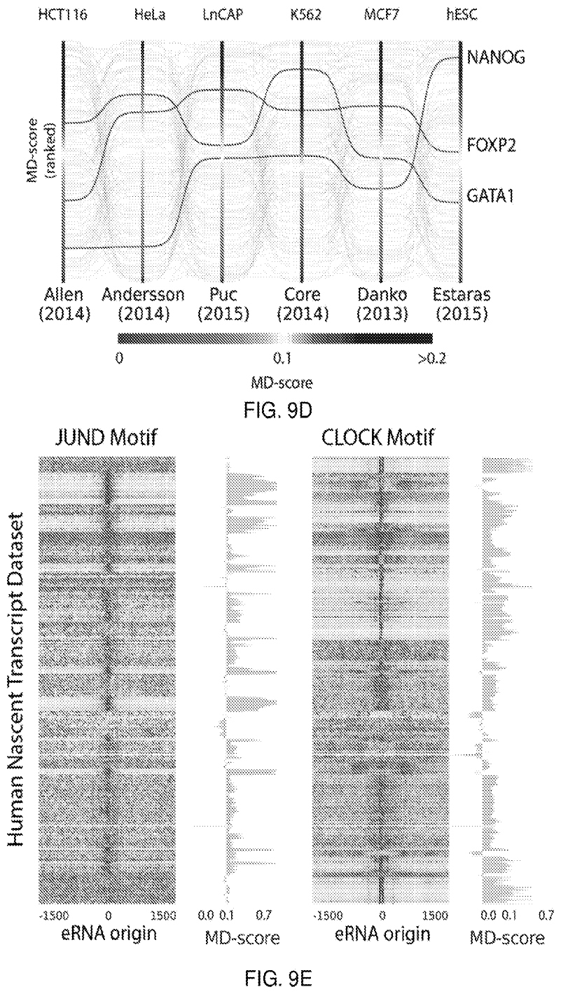

To further investigate the ability motif and eRNA co-occurrence to identify active TFs systematically requires a measurement the co-localization of motif instances with eRNA origination sites. With this in mind, a statistic was devised—the Motif Displacement score (MD-level)—which computes the proportion of TF sequence motif instances within an h—radius of eRNA origins relative to a larger local H-radius (see FIG. 9A, The Motif Displacement Score). Consistent with the average length of a nucleosome free region, the h—radius was set based on the average estimated distance between the forward and reverse strand transcript peaks at eRNA origins, and the H-radius as the average length of chromatin marks associated with active regulatory loci (h=150 bp, H=1500 bp; FIG. 8B). Consistent with the patterns observed in ChIP data, the MD-level is elevated in the bound set of eRNAs relative to the not bound set (FIG. 8C).

FIG. 8B is a histogram representing the distribution of estimated RNAP footprint size (distance between forward and reverse strand peaks) for Tfit predicted eRNAs (K562).

FIG. 8C is a plot indicating the co-association of instances of the motif with eRNA origin is elevated at bound sites. MD-level computed from x-axis: eRNAs that are not bound, and y-axis: TF-bound eRNAs.

FIG. 9A provides an example locus of GRO-seq, the inferred eRNA origin and computation of “motif displacement” and the associated MD-level.

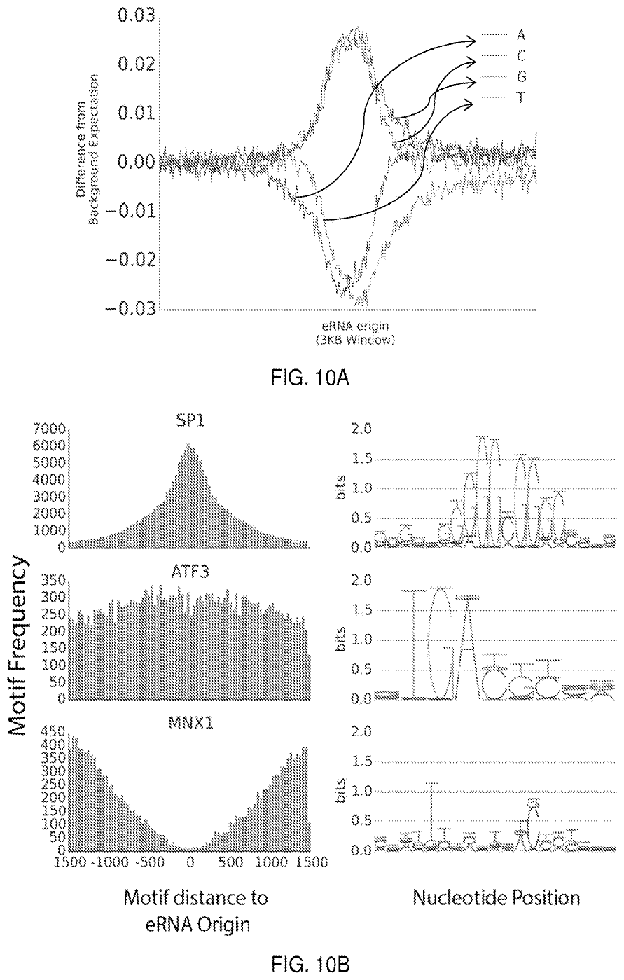

In order to expand this approach to include TFs for which no ChIP-seq is available, a hand-curated database of TF binding motif models (HOCOMOCO; Kulakovskiy et al., 2013, 641 motif models) was leveraged, and the distribution of motif instances proximal to K562 eRNA origins (FIG. 9B) measured. Under a uniform nucleotide background model, 32% of the motif models co-localized significantly with eRNAs (p-value<10⁻⁶; see The Motif Displacement Score). However, similar to gene promoters and TF binding motif instances, eRNAs (e.g. enhancers) exhibit heightened GC content, which may artificially induce GC-rich motif presence at eRNA origins (FIG. 10A). To control for local sequence bias in the co-localization metric, a simulation based method was developed to perform empirical hypothesis testing of the MD-level (see FIG. 10B and MD-level significance under a non-stationary background model). It was observed that—even in light of a significant nucleotide bias—27% of motif models remain significantly co-localized with eRNA origins in the K562 GRO-cap data set (FIG. 9C).

FIG. 9B is a heat map, where each row is a TF motif model and each column is a bin of a histogram (100) where heat is proportional to the frequency of a motif instance at that distance from an eRNA origin. FIG. 9C is a dot plot providing a comparison between the expected MD-level for a motif model (x-axis) and the observed MD-level in a K562 GRO-cap experiment[19]. Red and green dots indicate a p-value<10-6 above or below expectation hypothesis tests, respectively.

FIG. 10A indicates the position-specific bias surrounding eRNA predictions. eRNAs were predicted by Tfit from a K562 GRO-cap (SRR1552480) experiment and a 3 kb window (centered at eRNA origin) of sequence from the hg19 human genome build was collected. Background expectation was computed from the entire hg19 genome yielding 24.19%, 25.72%, 24.31%, 25.76% for A, C, T, and G nucleotides, respectively. 10⁹ 3 kb sequences were simulated from the empirical ACGT frequency depicted in FIG. 10A (FIG. 10B). The distribution of motif instances (significant PSSM matches <10⁻⁷) within simulated data is represented for three demonstrative transcription factors: SP1, ATF3 and MNX1 (as rows). Adjacent to each motif distribution, the associated PSSM in terms of information content (bits).

A subset of transcription factors display significantly lowered MD-levels relative to expectation, indicating that in these cases, the instances of the motif model are significantly depleted at eRNA origins. Consistent with this observation, a previously published knockout of the Rev-Erb family of repressors (Nr1d1 and Nr1d2) resulted in the gain of eRNAs. Taken together, these results indicate that repressors suppress eRNA activity proximal to their DNA response element.

Significant enrichment or depletion of a motif model near eRNA origins indicates that the transcription factor protein is present and functionally active, either as and activator or repressor, respectively. To validate that MD-levels reflect TF activity, the MD-levels of all motif models across a set of nascent transcript datasets from six distinct cell types were examined. Analysis revealed wide fluctuations in MD-levels of several motif models across experiments (FIG. 9D). Furthermore, the MD-level associated with cell type specific TFs are elevated in their known lineage of activity. For example, NANOG is elevated in embryonic stem cells consistent with its role in maintaining pluripotency. Additionally, GATA1 is elevated in K562 cells consistent with its role in leukemia.

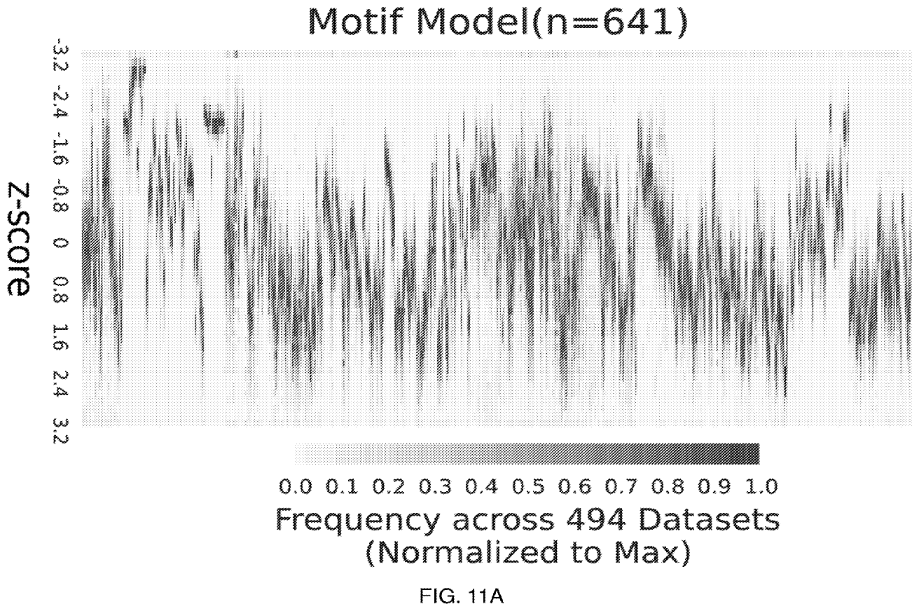

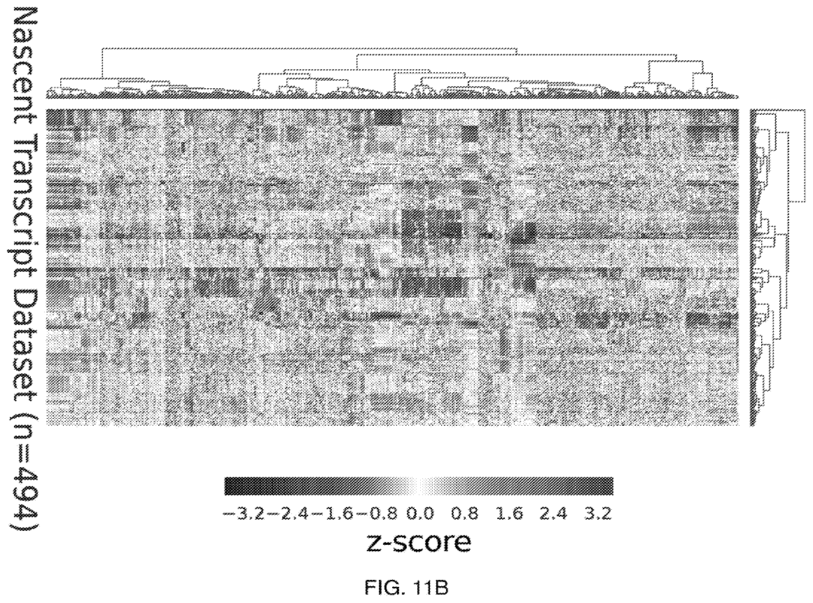

To further evaluate the MD-level, eRNA origins were predicted in a large collection of publicly available nascent transcription data sets (67 publications, 34 cell types and 205 treatments). The compendia included a diverse collection of nascent transcription protocols, cell types, sequencing depths, and laboratories of origin. Across the compendium, the spatial relationship between eRNA transcription and motif sequence is exceedingly dynamic (FIGS. 11A-11B), as exemplified by the JUND and CLOCK motif models (FIG. 9E). Given that a modest correlation between sequencing depth and eRNA-identification was observed, the extend to which the inferred MD-level score reflected batch effects was determined. The fact that many transcription factors play a pivitol role in cell fate and identity was leveraged. Dimensionality reduction of the MD-level compendium (491 human nascent transcription experiments) revealed statistical influences based prodeominantly on underlying cell type. Overall, 78% of motif models in HOCOMOCO are significantly co-localized with eRNA origins in at least one data set.

While the experimental details clearly influence the ability to infer specific eRNAs, the aggregation of genome-wide signal makes MD-levels robust to experimental variability. Key cell type specific transcription factors showed elevated MD-levels only in the relevant cell type (FIG. 9D), indicating that MD-levels quantify activity for broad classes of transcription factors across cell types, despite differences in protocol, sequencing depth and/or laboratory of origin. Overall, these results indicate that MD-levels fluctuate across cell types and conditions in a manner that suggests changes in transcription factor activity.

As an alternative validation, the transcription patters of the gene encoding the TF were examined. For many transcription factors, a higher transcription of the TF was observed when the MD-level significantly differed from expectation. Overall, 45% of TFs showed a correlation across all samples between the eRNA inferred MD-level and the transcription level (FPKM) of the gene encoding the transcription factor, indicating that some TFs are themselves regulated at transcription. However, the observed correlations were often weak and complex—typically neither linear or monotonic—consistent with the observation that expression levels of a gene are poorly correlated with protein levels. Many transcription factors, including TP53, are post-transcriptionally or post-translationally modified to regulate their activity and therefore FPKM and MD-levels are not expected to correlate.

FIG. 9D is a diverging color map, which ranges from [0, 0.2], indicating MD-levels computed and ranked under six nascent transcript data sets. FIG. 9E is a pair of heat maps, where each row corresponds to a nascent data set and each column relates to motif frequency. These motif displacement distributions are are shown for two demonstrative examples (JUND and CLOCK) and the associated MD-levels, sorted by publication.

The heat maps of FIGS. 11A-11B demonstrate that MD-levels display wide variability across all publicly available nascent transcript datasets. Sites of bidirectional transcription were profiled by Tfit across the full compendium of nascent transcription data sets allowing computation of the 641 (HOCOMOCO) MD-levels. In FIG. 1A, each row is a single motif model, plotted as the histogram of z-scores (MD-levels were centered by the mean and scaled by the standard deviation). In FIG. 1B, each row represents a motif model and each column represents a nascent transcription data set. Heat indicates higher MD-levels (relative to the mean). Rows and columns were separately sorted by hierarchical clustering.

### Motif Displacement Scores Quantify TF Activity

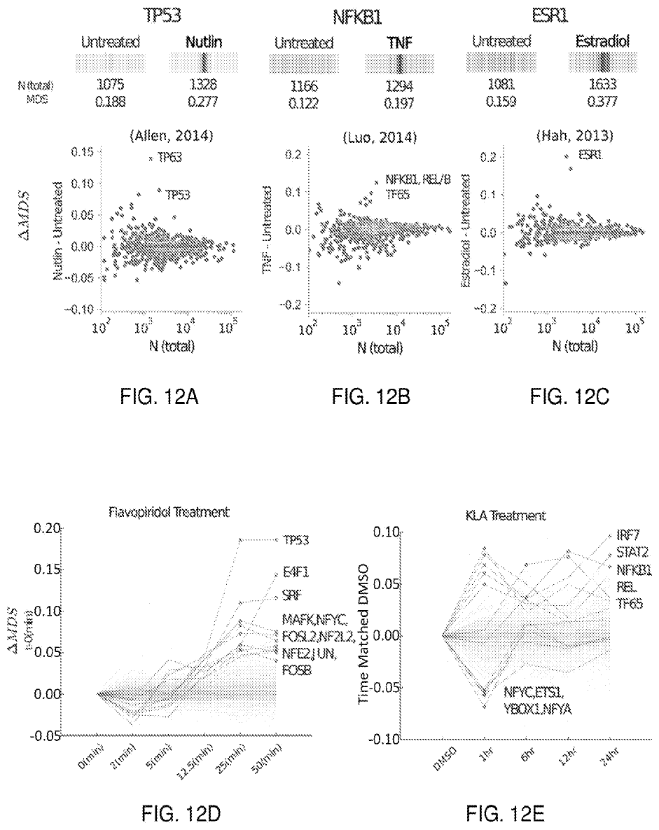

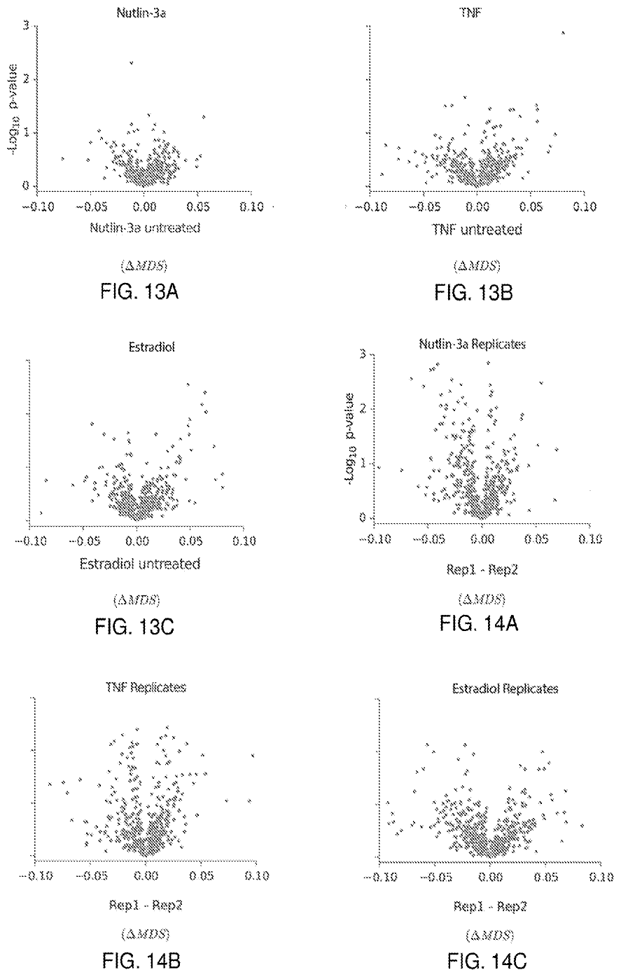

To better investigate whether MD-levels reflect TF activity, experiments where the activity of individual TFs is perturbed were examined. It was reasoned that alterations in TF activity should be detected as significant changes in the MD-level. The drug Nutlin-3a has been used to activate TP53 in HCT116 cells (Allen et al., 2014, eLife, 3:e02200). A significant increase in the co-localization of the TP53 motif sequence and eRNA origins following one hour of Nutlin-3a exposure (AMD-level 0.17, p-value<10⁻³³) was observed in the present study. Of the 641 available TF-motif models, only TP53 and TP63—which have nearly identical motif models, displayed elevated MD-levels following Nutlin-3a treatment (p-value<10⁻⁶, FIG. 12A). Differential MD-level analysis between biological replicates revealed no significant shifts in motif to eRNA co-localization, indicating that the false discovery rate is low (FIG. 13A).

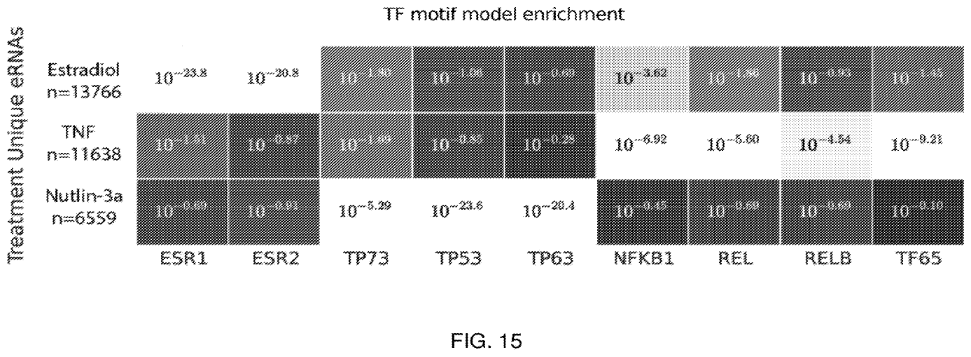

A number of other studies have specifically activated TFs including tumor necrosis factor (TNF, also known as TNF-α) activation of the NF-κB complex (NFKB1/NFKB2/REL/RELA/RELB) and estradiol activation of ESR1. Even in light of distinct nascent transcription protocols, dramatic shifts were observed in the MD-level for the transcription factor(s) known to be activated by each stimulus (FIGS. 12B-12C). As before, differential MD-level analysis between biological replicates revealed no significant shifts in motif-eRNA co-localization (FIGS. 13B-13C). Despite that treatments involving Nutlin-3a, TNF, and estradiol are known to modulate gene expression, no detectable differences in MD-levels were observed when considering only promoter-associated bidirectional transcript sites (FIGS. 14A-14C). In all three cases (FIGS. 12A-12C), TF activation resulted in the production of new eRNAs that are uniquely enriched for the relevant motif model, effectively elevating the TF's MD-level (FIG. 15).

FIGS. 12A-12C demonstrate that MD-levels are predictive of TF activity. The top panel of FIG. 4A indicates the motif displacement distribution, MD-level, and the number of motifs within 5 kb of any eRNA origin before and after stimulation with Nutlin-3a (e.g., Nutlin) on TP53, the transcription factor known to be activated. The bottom panel of FIG. 4A indicates the change in MD-level (ΔMDS) following perturbation (y-axis) relative to the number of motifs within 1.5 kb of any eRNA origin (x-axis). Red points indicate significantly increased and/or decreased MD-levels, respectively (p-value<10⁻⁶). Similar analysis for TNF activation of the NF-κB complex and Estradiol activation of estrogen receptor (ESR1) are illustrated by FIGS. 12B and 12C, respectively. (D) A time series data set following treatment with Flavopiridol Jonkers2014. The y-axis indicates the MD-level change relative to time point 0. Blue dots indicate a MD-level difference <10⁻⁶. A darker shaded line indicates a time trajectory with at least one significant MD-level. (E) Time series data set following treatment with Kdo2-lipid A (KLA) where each time point is normalized to time-matched DMSO Kaikkonen2014. Therefore, the y-axis indicates MD-level difference relative to the time point matched DMSO sample.

The plots of FIGS. 13A-13C illustrate that no significant differences in MD-levels were found between biological replicates. Experiments annotated as biological replicate pairs: (SRR1105738, SRR1105739), (SRR1015589, SRR1015590), (SRR653425, SRR653426) were used to study differences in MD-levels for Nutlin-3a (FIG. 13A), TNFα (FIG. 13B) and estradiol (FIG. 13C), respectively. Y-axis indicates the negative log 10 p-value (two-tailed proportion test) in MD-level change. The x-axis provides the change in MD-level.

The plots of FIGS. 14A-14C illustrate that no significant differences in MD-levels were found when considering only promoter associated bidirectional transcripts. Experiments annotated as treatment/control pairs: (SRR1105737, SRR1105739), (SRR1015583, SRR1015589), (SRR653421, SRR653425) were used to study differences in MD-levels following treatment for Nutlin-3a (FIG. 14A), TNFα (FIG. 14B) and estradiol (FIG. 14C), respectively. MD-levels were computed over promoter associated bidirectional transcripts. Y-axis indicates the negative log 10 p-value (two-tailed proportion test) in MD-level change. The x-axis provides the change in MD-level.

The table of FIG. 15 indicates that the treatment-unique eRNAs are enriched for specific TF binding motif instances. Treatment-specific eRNAs were isolated from experiments annotated as treatment/control pairs: (SRR1105738, SRR1105739), (SRR1015589, SRR1015590), (SRR653425, SRR653426) for Nutlin-3a, TNFα and estradiol respectively. A treatment-unique eRNA is considered if the eRNA origin is not within 1500 base-pairs of control-present eRNA. A treatment-unique eRNA is considered associated with a motif if the motif center is within 150 base pairs of the eRNA origin. Significance of motif over representation is assessed via a one-tailed hypergeometric distribution. Cell color is proportional to the log 10 of the p-value.

The robustness of the ΔMD-level approach for inferring altered TF was evaluated. Firstly, differential MD-level analysis between biological replicates revealed no significant shifts in motif sequence to eRNA co-localization, indicating a low false discovery rate. Secondly, reads from the Nutlin-3a experiment were randomly subsampled to generate data sets with considerably lower depth. With increasingly less depth, fewer eRNAs were detected and the inferred MD-level dropped. However, the magnitude of the ΔMD-level remained relatively consistent, indicating that the metric is largely robust to sequencing depth. Finally, the h-radius was varied from 0 to 1500 base-pairs (the full H-radius) to assess the impact of the h-radius on differential MD-level analysis. Detectable differences in MD-level were found across a broad range of h-radius values, indicating that detection of significant ΔMD-level is robust to the choice of h-radius. Collectively, these results indicate that differential MD-level analysis is a robust method of detecting changes in TF activity.

In each of the aforementioned perturbations, nascent transcription was assessed at a time point of one hour or less. Therefore, it was determined whether MD-levels could capture transcription factor activity across broader time frames. First, it was observed that detectable changes in TF activity are exceedingly rapid, as exemplified by flavopiridol (a CDK9 inhibitor) treated mouse embryonic cells which display a dramatic and monotonic increase in the MD-level of TP53 and E4F1 (FIG. 12D). For a number of TFs, MD-levels trend upward at 12.5 minutes and show significant changes within 25 minutes of exposure. This result indicates that eRNA activity proximal to key TFs increases at short time points, even though flavopiridol is a general repressor of transcription. Mouse T-cells treated for a longer time course with Kdo2-lipid A (a highly specific TLR4 agonist) treated mouse T-cells, showed dynamic and time-ordered shifts in MD-levels for a number of key transcription factors (FIG. 12E), including interferon (IRF7) and STAT2. Furthermore, YBOX1 decreased in co-localization (reduced MD-level), consistent with its known role as a transcriptional repressor that increases in expression after KLA exposure. Collectively, these results indicate that profiles of eRNA transcription—when combined with motif models—identity shifts in TF activity in response to perturbation.

FIG. 12D data set following treatment with Flavopiridol Jonkers2014. The y-axis indicates the MD-level change relative to time point 0. Blue dots indicate a MD-level difference of <10⁻⁶. A darker shaded line indicates a time trajectory with at least one significant MD-level.

FIG. 12E presents a time series dataset following treatment with Kdo2-lipid A (KLA) where each time point is normalized to time-matched DMSO. Therefore, the y-axis indicates MD-level difference relative to the time point matched DMSO sample. GEO SRR numbers of these comparisons are outlined in Table 2.

TABLE 2
Nascent Transcript Data Set Usage in Pairwise Comparisons

| FIG. | | |
|---|---|---|
| Number | Group A | Group B |
| 12A | SRR1105737 | SRR1105739 |
| 12B | SRR1015583 | SRR1015587 |
| 12C | SRR653421 | SRR653425 |
| 12D | SRR935093 | SRR935093 (none) |
| | | SRR935097 (2 minutes) |
| | | SRR935101 (5 minutes) |
| | | SRR935105 (12.5 minutes) |
| | | SRR935109 (25 minutes) |
| | | SRR935113 (50 minutes) |
| 12E | SRR930649(DMSO; 1 | SRR930659 (KLA; 1 hour) |
| | hour) | |
| | SRR930653(DMSO; 6 | SRR930663 (KLA; 6 hour) |
| | hour) | |
| | SRR930655(DMSO; 12 | SRR930665 (KLA; 12 hour) |
| | hour) | |
| | SRR930657(DMSO; 24 | SRR930667 (KLA; 24 hour) |
| | hour) | |
| 16A | SRR1145801 | SRR1145801 (hESC) |
| | | SRR1145808 (endoderm) |
| | | SRR1145815 (primitive gut tube) |
| | | SRR1145822 (posterior foregut) |
| | | SRR1145829 (pancreatic |
| | | endoderm) |
| 16B | SRR1552482 | SRR1552480 |
| | SRR1552483 | SRR1552481 |
| | SRR1552485 | SRR1554311 |
| 16C | SRR639050 | SRR1041870 |
| | SRR014286 | SRR1041871 |
| | | SRR408117 |

### MD-Levels Predict TF Activity Across Cell Types

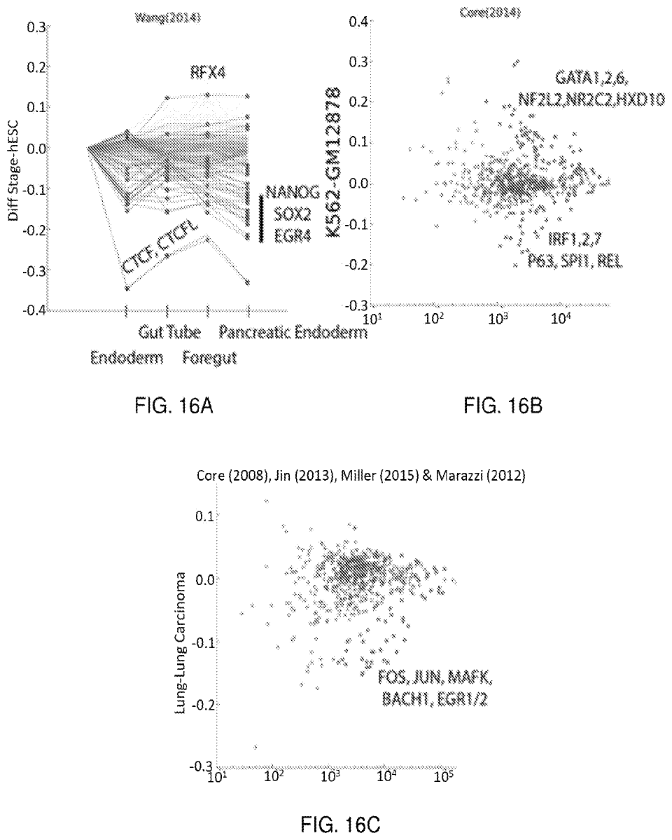

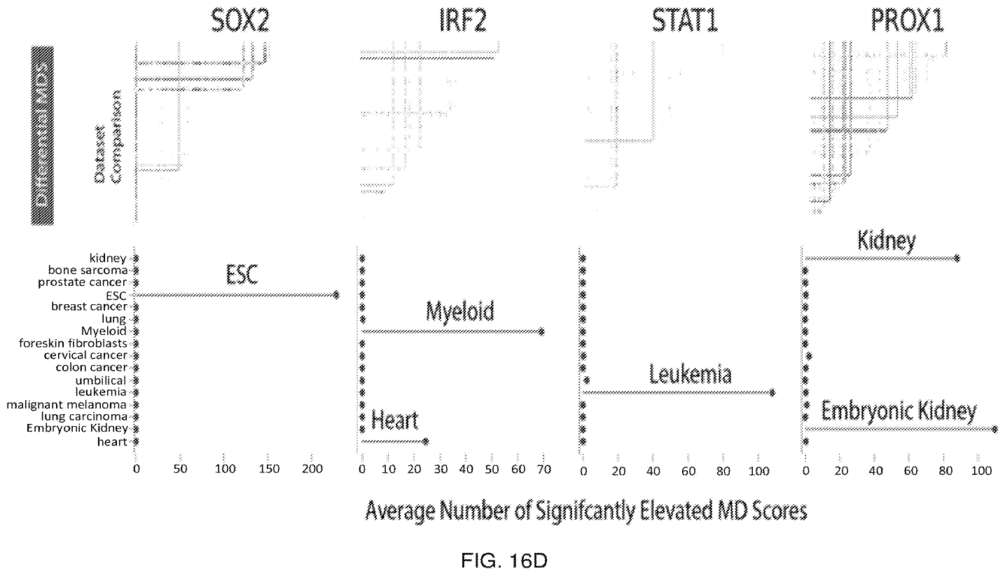

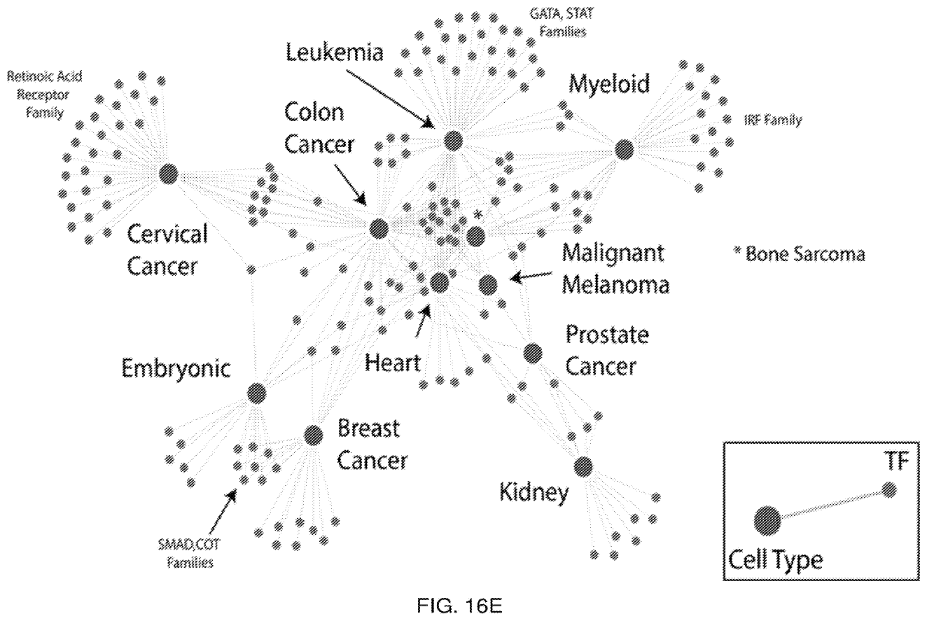

Stimulus responsive TF activity is detectable by motif displacement over eRNA origins, but transcription factors also play a pivotal role in cell fate and identity. With this in mind, a differentiation time series was examined where human embryonic stem cells were differentiated into pancreatic tissue. In this scenario, a substantial decrease in MD-level was observed for transcription factors OCT4, SOX2, P052 and NANOG immediately following differentiation to endoderm, concordant with their role as embryonic stem cell markers (FIG. 16A). Furthermore, RFX4, which has the same motif as the pancreatic islet specific RFX6, is elevated late in the time series.

It was then determined whether differential MD-levels could be utilized to identify, without prior knowledge, the key TFs that define distinct cell types. An examination of eRNA/motif co-localization between two different cell types—GM1278 (Myeloid) and K562 (Leukemia) cell lines—resulted in higher activity of the GATA family of transcription factors in leukemia (K562) cells, whereas the interferon-regulatory family of TFs were more active in Myeloid (GM1278) cells (FIG. 16B). When comparing more closely related cells, specifically fetal-lung tissue (IMR90) and lung carcinoma (A549), a strong increase in the transcription factors related to the BACH1/MAFK pathway in the lung carcinoma samples (FIG. 16C) was noted.

The plot of FIG. 16A indicates MD-level changes following differentiation of human embryonic stem cells to pancreatic endoderm. FIGS. 16B-16C provide comparisons of MD-level changes between different cell types. GEO SRR numbers of these comparisons are outlined in Table 2.

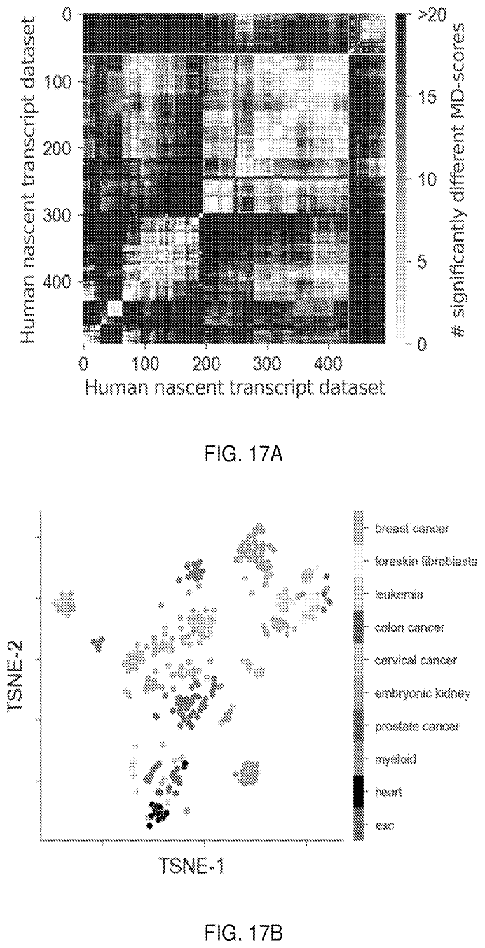

To move beyond isolated pairwise comparisons, MD-levels between all possible human, untreated dataset pairs (124,251 pairwise comparisons, FIGS. 17A-17B) were examined. Cell type-matched datasets revealed lower numbers of significantly altered MD-levels than cell type-differing comparisons, indicating a cell type effect on motif/eRNA association (FIGS. 13A-13B). Indeed, the SOX2 motif model, a well-established cell type-specific TF, differentially co-occurs with eRNAs in 7,554 comparisons, 99.8% of which are embryonic stem cells (FIG. 16D). Following in suit, motif models for IRF2, STAT1 and PROX1 were significantly enriched

The MD-levels presented in FIGS. 17A-17B were computed for all human nascent transcript data sets in the compendium. FIG. 17A presents a pairwise comparison of each data set shown as a distance matrix where each cell's heat is proportional to the number of significantly different MD-levels (p(ΔMDS≠0)<10⁻⁶). Rows and columns are sorted by Ward hierarchical clustering via Euclidean distance metric. FIG. 17B indicates dimensionality reduction by t-Distributed Stochastic Neighbor Embedding (TSNE) of the distance matrix in panel A. Only publication annotated cell types with at least ten data sets are shown, each data set is a point.

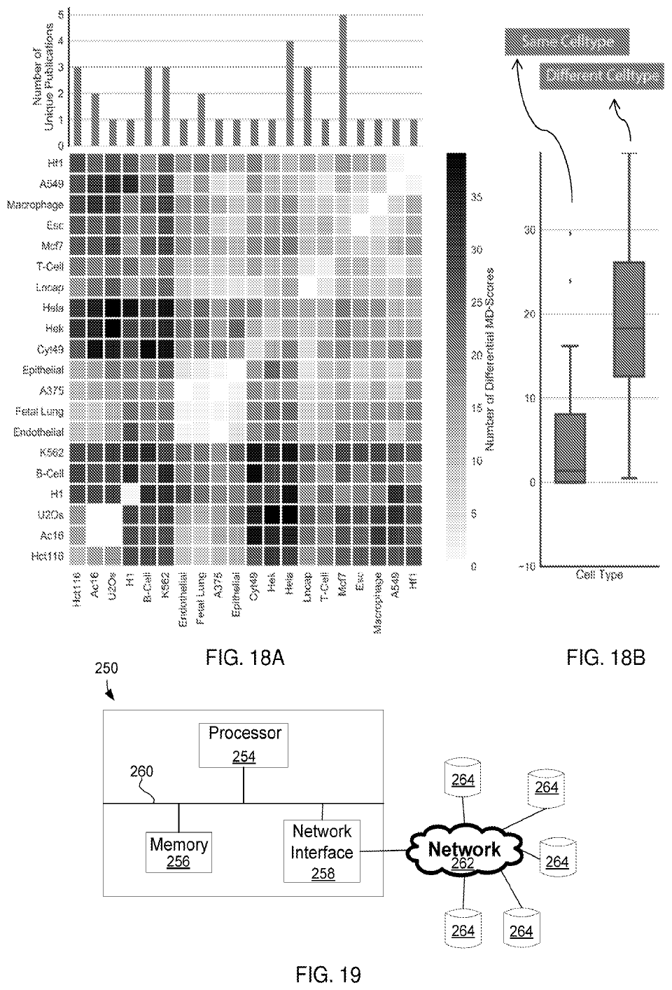

For the data presented in FIGS. 18A-18B, each untreated nascent transcript dataset (n=158) was independently compared and assessed for significant changes in MD-levels (possible comparisons=12403). FIG. 18A presents a heatmap indicating the average number of significantly altered MD-levels (p-value<10⁻⁶) between any two experiments that are annotated as the associated cell type. FIG. 18B indicates the distribution of the number of significantly different MD-levels grouped by comparison type: same (e.g. ESC to ESC) or different (e.g. HeLa vs LnCAP) cell type. Hypothesis testing on the means of these distributions was performed by the standard t-test. Specific to FIG. 18B, comparisons were only made if the data sets were from different publications. FIGS. 18A and 18B indicate that fewer MD-levels are significantly different within similar cell types than across different cell types.

Each rotated matrix of FIG. 16D provides a comparison between all possible pairs of datasets. Blue shading represents a significant deviation of the ΔMDS from 0 (p-value<10⁻⁶) from experiment i and j.

Finally, cell type-specific TF activity across all tissue types for which nascent transcription data is available was identified (FIG. 16E). In FIG. 16E, a blue node indicates a TF while a green node indicates a cell type. An edge is drawn if the enrichment score's p-value falls below 10⁻⁶. Examination of the co-association network reveals many well-documented links between cell type and TF activity (e.g. retinoic acid receptors & cervical cancer). Even still, this analysis uncovered dozens of previously unknown cell type-unique transcription factors whose mechanistic contribution to cellular identity have yet to be investigated. In addition to cell type-unique associations, this analysis also reveals inter-cell type relationships: MCF7 breast cancer and embryonic stem cells share activity of COT and SMAD family TFs, consistent with recent evidence linking stem cell-like behavior to breast cancer. As the diversity and quantity of nascent transcription data increases, eRNA profiling will precisely define the biological systems where individual TFs exert their regulatory influence.

### Discussion

The observation that eRNAs mark the functional activity of transcription factors was leveraged to develop a statistic that reflects a transcription factor's functional activity. Importantly, TFs are not assigned to individual enhancers, because most eRNAs have numerous motif instances proximal to their origin. The approach presented herein does not determine which of these possibilities is critical to the regulation of the eRNA. Rather, the statistic, the MD-level, measures the co-localization of eRNAs with a TF motif model in order to capture changes in TF activity after diverse stimuli.

While the biological functions of eRNAs remain largely unknown, as presented herein, eRNAs clearly represent a powerful readout for TF activity.

Separately, it has been noted that some binding sites are apparently ‘silent’ with respect to transcription or reflect artifacts of ChIP. Therefore, to determine whether eRNAs mark sites of TF activity, binding events were leveraged across cell lines that differed only in their eRNA activity. The results indicate that TF binding sites that correspond to eRNA synthesis are more likely to positively affect nearby gene expression than those lacking eRNA transcription. Undoubtedly, assigning enhancers to the nearest gene is not optimal, as many enhancers are known to regulate target genes at great distances. However, incorrect enhancer-to-gene assignments would only increase noise within the comparison. Thus, given the instability and short half-lives of eRNAs, their presence within a cell reflects ongoing TF activity.

Consequently, TF activity was directly assessed from motif models and nascent transcription. It was observed that many motif models show significantly enriched co-localization with eRNA origins beyond expectation, indicating that these TFs are both present and functionally active in regulation. It was demonstrated that TF activity is a strong predictor of cell type, even across distinct protocols, sequencing depths, and laboratory of origin. Hence, the approach of the present disclosure has utility in identifying diagnostic signatures of transcription factor activity.

Further, MD-levels can be used to identify when the activity of a TF differs between two data sets, either due to an experimental stimulus or differences in cell type. The MD-level metric utilizes the genome-wide patterns of TF motif sequence co-localization with eRNA origins to identify changes in TF activity, regardless of whether the TF functions as an activator or repressor. Implicitly, changes in MD-level must thus reflect the gain and loss of eRNAs between two conditions, suggesting a direct relationship between functional TF binding and eRNA transcription initiation.

Addition of diverse chemical stimuli to cells resulted in activation or deactivation of specific TFs. The present approach may serve as a screen capable of discriminating between the direct mechanistic impact of closely related compounds, and hence serve as another layer of information about the effects of a drug. The present approach may also provide a simple screen for how mutations within TFs result in altered molecular profiles, and how such profiles contribute to human disease.

### Materials and Methods

### Public Data Sets

The relationship (association and/or overlap) between genomic features such as TF binding peaks, chromatin modifications, DNA sequence, TF binding motif models and enhancer RNA presence is examined in the Examples of the disclosure. Data for all features was obtained from publicly available sources and compared relative to a human and mouse genome, versions hg19 and mm10, respectively Human and mouse nascent transcription data was obtained from the NCBI Gene Expression Omnibus. ENCODE peak data was obtained from encodeproject.org/matrix/?type=Experiment. Most data was provided relative to hg19, but when necessary, ENCODE files were converted to hg19 via the Python liftover package. Motif models were obtained from the HOCOMOCO v.10 database and scanned against the genome.

eRNA Origins

In prior work (Azofeifa and Dowell, 2017) the known behavior of RNA polymerase II was leveraged to identify individual transcripts within nascent transcription data. Although the model, known as Transcription fit (Tfit; Azofeifa and Dowell, 2017), does not implicitly assume polymerase initiation will be bidirectional, bidirectional transcription was observed at both promoters and enhancers. Whether bidirectional (2 transcripts) or unidirectional (1 transcript), the Tfit model precisely infers the point of RNA polymerase loading, e.g. the origin point of transcription. Formally, this origin point (μ) represents the expected value of a Gaussian (Normal) random variable discussed in great detail in Azofeifa and Dowell (2017) and later in the present disclosure.

As reported previously (Azofeifa and Dowell, 2017), it was observed that super enhancers and regions annotated as transcribed often contain multiple origins. For example, as shown in FIG. 2, the entirety of this super enhancer annotated region is transcribed and identified as a single region by most nascent transcription analysis algorithms. However, since Tfit looks for site of initiation rather than transcribed regions, the Tfit model identifies three origins of transcription in this region, each giving rise to two transcripts proceeding in opposite directions. For clarity, the region is referred to as a “transcribed region”, each inferred position of polymerase initiation as an eRNA origin, and the resulting transcripts (in this case six) as individual eRNAs.

### Genomic Feature Data Integration

The relationship between genomic features such as TF binding peaks, chromatin modifications, DNA sequence, TF binding motif instances and enhancer RNA presence was examined. Frequently, two (or more) datasets were compared for association between the genomic features. Unless otherwise stated, two genomic features are said to overlap or associate if the two elements are located on the same chromosome and the center of their feature is within 1500 base pairs of each other. For example, let some TF binding peak be located on chromosome 1 with a start coordinate of 10000 and stop coordinate of 10405 and an eRNA origin at chromosome 1 with a start coordinate of 10200 and stop coordinate of 10201. Given that the center of TF binding peak is ((10405+10000)/2=10202.5) and 110202.5-10200.51<1500, these two genomic coordinates are considered to associate. Furthermore, all genomic coordinates refer to hg19 or mm10 for human or mouse datasets, respectively.

### Validation of Revised Tfit Algorithm

Prior work, including the earlier version of Tfit (Azofeifa and Dowell, 2017), has demonstrated a tight relationship between bidirectional transcription and enhancer associated histone marks. Using the modified Tfit, DNase I hypersensitivity (DHS1), histone 3 lysine 27 acetylation (H3K27ac) and histone 3 lysine 4 mono-, di-& tri-methylation significantly associate with non-promoter bidirectional transcription, as expected (FIG. 1). Indeed, histone modifications are displaced from bidirectional centers (origins) supporting the presence of a nucleosome-free region localized precisely at the origins of bidirectional transcript initiation (FIG. 1B). As reported previously (Azofeifa and Dowell, 2017), it was observed that super enhancers and regions annotated as transcribed often contain multiple origins. For example, as depicted by FIG. 2, the entirety of this super enhancer annotated region is transcribed and identified as a single region by most nascent transcription analysis algorithms (e.g., Allison et al., 2013; Azofeifa et al., 2014; Chae et al., 2015). However, since Tfit looks for sites of initiation rather than transcribed regions, the model identifies three origins of transcription within this region, each giving rise to two transcripts proceeding in opposite directions. For clarity, the region is referred to as a “transcribed region”, each inferred position of polymerase initiation as a bidirectional, regardless whether one or two transcripts are produced. If the site of polymerase initiation is not promoter associated, it is referred to as an eRNA origin, and the resulting transcripts (six are observed in FIG. 1) as individual eRNAs.

Depth of Sequencing on eRNA Inference

As the sequencing depth of each experiment varied dramatically, it was desired to understand the relationship between depth and eRNA inference. To this end, eRNAs were inferred across the complete compendium of human data sets (491 experiments) and the number of eRNA origins predicted by Tfit was plotted against the depth of sequencing. The resulting contour plots indicated a weak but present correlation in the number of bidirectionals inferred (in any experiment) relative to the underlying sequence depth of that experiment. In other words, more depth predicts a slightly higher rate of bidirectional (or eRNA origins) inference.

Characterization of eRNA Inference: Comparison to ChIP

Prior work observed a significant overlap between eRNA predictions and transcription factor binding data. This observation is confirmed using the eRNAs called by Tfit. To this end, the set of eRNA origins is integrated with the genomic binding locations of 139 proteins profiled by ChIP-seq, also in K562 cells. Consistent with previous results, 98% of eRNAs are bound by at least one regulator, where an average of 52.9 regulators localize at any one eRNA (FIGS. 5A and 5B). In fact, three distinct patterns of TF binding are observed (FIG. 1C): TFs that bind all eRNAs (32 factors co-occur with over 75% of all eRNAs; clade IV); TFs that bind only a few eRNAs (39 factors associate with no more than 20% of all eRNAs; clades I & II); and TFs that bind to many eRNAs but only with unique TF partners (58 factors occur under specific combinatorial patterning, e.g. GATA2/NR2F2/GABPA and FOSL/ATF3 strongly co-localize at eRNAs; clades III & V). TF-combinatorial control also plays a pivotal role in downstream gene regulation. In general, the number of TFs co-localized at sites of open chromatin is larger when an eRNA is present than not (FIG. 1E). Furthermore, TF co-association dramatically increases when considering eRNA presence (FIG. 1F). In summary, unique sets of TFs bind to specific eRNA origins.

The co-localization of TF binding relative to eRNA origins was examined (FIG. 1D). Two classes of regulators were observed: 84% of TFs exhibit centered, unimodal localization with eRNA origins and 16% display significantly displaced peak localization flanking eRNA origins. For example, factors such as RBB5, PHF8 and CDH1 are significantly displaced an average of 150, 200, and 398 bp from the eRNA origin, respectively (FIG. 5C). Regulators with displaced peak localization are significantly enriched for ontological definitions such as “histone modification,” “chromatin organization” and “histone deacetylation” consistent with the bimodal distribution of histone modifications observed in Supplemental Fig. Error! Reference source not found.B (p-value<10⁻⁶).

### Nascent Transcription Data Processing

SRA files were downloaded from the NCBI Gene Expression Omnibus (GEO, available online at ncbi.nlm.nih.gov/geo/). The SRA files were converted into fastq format using fastq-dump 2.3.2-5 in the SRA Toolkit. Studies utilizing a second strand synthesis kit were reverse complemented using fastx-reverse-complement (Halbritter, 2013, Geneprof manual)-Q33 Human and mouse fastx files were mapped to the hg19 and mm10 genomes, respectively, using bowtie2 (Langmead and Salsberg, 2012, Nat Meth, 9:357-59) version 2.0.2. The resulting sam files were converted to sorted bam files using samtools (Li et al., 2009, Bioinformatics, 25:20178-79) version 0.1.19. Each sorted bam was converted into two strand-separated bedgraphs (one file containing positive strand and one with negative strand reads) using bedtools (Quinlan and Hall, 2010, Bioinformatics, 26:841-42) genomeCoverageBed version 2.22.0. The hg19_all.fa genome file from UCSC was used for human data and mm10 Bowtie2 index.fa for mouse data. The bedgraphs were sorted then converted to bigwig format using bedGraphToBigWig (Kent et al., 2010, Bioinformatics, 26:2204-07). The hg19.chrome.sizes and mm10.chrome.sizes input files were made using fetchChromSizes (Tan, 2016, Cne identification and visualization) from UCSC and the hg19 and mm10 genome files, respectively.

### Tfit Modification and Parameters

Transcription fit (Tfit) is a finite mixture model that utilizes a model of RNA polymerase II behavior to identify and characterize all transcripts in nascent transcription data (Azofeifa and Dowell, 2017). The known behavior of RNA polymerase II is leveraged to identify individual transcripts within nascent transcription data. The Tfit model infers the precise point of RNA polymerase loading; e.g., the origin point of transcription. Formally, this origin point (μ) represents the expected value of a Gaussian (Normal) random variable.

For analysis of numerous nascent data sets as described in the present disclosure, this previous approach is modified in two ways. First, to rapidly identify all sites of transcription initiation genome-wide, a likelihood ratio statistic between a fully specified exponentially-modified Gaussian (equation, the loading/initiation/pausing phase of the earlier Tfit model) against a Uniform distribution background model (Equation equation 2) at some genome interval [a,b] was computed. This approach is hereafter referred to as Template Matching. Second, the earlier estimate of the loading step of polymerase activity is amended to permit variable distances between the forward and reverse strand transcripts, hereafter referred to as a polymerase footprint. Both modifications are described in full detail below.

### Template Matching

The loading/initiation/pausing portion of the Tfit model, fully specified in Azofeifa and Dowell, 2017, describes the initial activity of RNA polymerase II (RNAP) and captures initiating transcription, which is often bidirectional, genome-wide. The model assumes RNAP is first recruited and binds to some genomic coordinate X as a Gaussian distributed random variable with parameters μ,σ² where μ might represent the typical loading position (e.g. origin of any resulting transcript either TSS or enhancer locus) and σ² the amount of error in recruitment to μ. Upon recruitment, RNAP selects and binds to either the forward or reverse strand, which is characterized as a Bernoulli random variable S with parameter π. Following loading and pre-initiation, RNAP immediately escapes the promoter and transcribes a short distance, Y. It is assumed that the initiation distance, is distributed as an exponential random variable with rate parameter In this way, the final genomic position Z of RNAP is a sum of two independent random variables (X+SY) where the density function (resulting from the convolution/cross-correlation) is given in equation. In keeping with traditional notation, upper case, non-greek alphabet letters represent random variables and the associated lower case refer to instances or observations of the stochastic process.

$$h ( z , s ; μ , σ , λ , π ) = λ φ ( \frac{z - μ}{σ} ) R ( λσ - s \frac{z - μ}{σ} ) {h}_{} ( s ) \\ {h}_{} ( s ) = \{ \begin{matrix}π \text{:} & s = + 1 \\ 1 - π \text{:} & s = - 1\end{matrix} \qquad ( equation 1 )$$

In equation 1, R(⋅) refers to the Mill's ratio and (p(⋅) refers to the standard normal density function.

In contrast, reads obtained outside of initiation regions are captured by a Uniform distribution (equation equation 2):

$$u ( z ; a , b ) = \frac{\hat{π}}{b - a} , \qquad ( equation 2 )$$

where π refers to the maximum likelihood estimator for the strand bias (equation equation 3):

$$\hat{π} = {\sum }_{i = 1}^{N} I ( {s}_{i} > 0 ) / N \qquad ( equation 3 )$$

Where I(⋅) is an indicator function

Finally, the (log-)likelihood of the exponentially modified Gaussian (emg) and Uniform (u) distribution computed at a genomic interval [a,b] using aligned read counts is given in equation 4:

$${LL}_{emg} = {\sum }_{i = a}^{b} log h ( {z}_{i} , {s}_{i} ; \hat{μ} , \hat{σ} , \hat{1} / λ , \hat{π} ) \\ {LL}_{u} = {\sum }_{i = a}^{b} I ( {s}_{i} > 0 ) log \frac{\hat{π}}{b - a} + I ( {s}_{i} < 0 ) log \frac{1 - \hat{π}}{b - a} \\ LLR = {LL}_{emg} - {LL}_{u} . \qquad ( equation 4 )$$

In equation 4,\hat{μ} refers to the center of the window. Based on a previous study, {\hat{σ}, \hat{ }l/λ,ŵ,\hat{π}}={34.2,391.7,0.358,0.501}.

The algorithm is a sliding window of LLR computations. Overlapping (1 base pair) regions of interest (LLR>τ) are merged. In every study profiled for bidirectional transcription by Tfit, τ=10³. τ is estimate from the False Discovery rate analysis of predicting TF binding events from bidirectional transcription alone. More information on running and using Tfit output is available at github.com/azofeifa/Tfit.

### EM Algorithm and Bidirectional Origin Estimation

On its own however, the template matching module of Tfit does not provide an exact estimate over i (the parameters associated with a single loading position). To perform optimization over τ and specifically μ (the origin of bidirectional transcription), the Expectation Maximization algorithm (outlined in detail in Azofeifa and Dowell, 2017) was derived to optimize the likelihood function of equation. The following EM-specific parameters were used at each loci: the number of random re-initializations per loci was set to 64, the threshold at which the EM was said to converge, |llₜ−llₜ₊₁|, was set to 10⁻⁵. For computational tractability, the EM algorithm halted after maximum of 5000 iterations.

At each window predicted by the sliding window algorithm, inference over μ, σ, λ, and π is performed by the EM algorithm. Details of the derivation, model selection and algorithm design can be found in Azofeifa and Dowell, 2017.

### Footprint Estimation

Previous efforts at parameter estimation of the finite mixture model assumed that RNA polymerase II behaved as a point source (Azofeifa and Dowell, 2017). Consequently, a systematic approach could not be incorporated to estimate observed gaps between the forward and reverse strand peaks which deviate more than could be explained by an exponentially-modified Gaussian density function. The model is amended to estimate this behavior. The distance between the forward and reverse strand peaks is termed the footprint of RNA polymerase II or fp. fp amounts to adding or removing a constant to zᵢ. Assuming that fp>0 then the above equations remain valid by a transformation to zᵢ.

zᵢ:=zᵢ−sᵢ·fp

As in Azofeifa and Dowell, 2017, this new parameter is inserted into the conditional expectation of the latent variables given the observed random variables and perform a gradient step. This allows optimization for fp (equation equation 5).

$$\hat{f} {p}_{k} := \frac{1}{{r}_{k}} {\sum }_{i = 1}^{N} ( {s}_{i} ( {z}_{i} - μ ) - E [ Y \vert {z}_{i} , {s}_{i} ; {θ}^{g} ] ) \cdot {r}_{i}^{k} \qquad ( equation 5 )$$

Derivation of the EM algorithm and fitting the Tfit model are discussed heavily in Azofeifa and Dowell, 2017. The full expression of the expectation operators is given in equation 6.

$$E [ Y \vert {g}_{i} ; {θ}^{t} ] = {s}_{i} ( z - μ ) - λ {σ}^{2} + \frac{σ}{R ( λ σ - {s}_{i} ( {z}_{i} - μ ) / σ )} \\ {r}_{i}^{k} = p ( k \vert {g}_{i} ; {θ}_{k}^{g} ) = \frac{{w}_{k} \cdot p ( {g}_{i} ; {θ}_{k}^{g} )}{{\sum }_{k ∈ K} {w}_{k} \cdot p ( {g}_{i} ; {θ}_{k}^{g} )} \\ {r}_{k} = {\sum }_{i = 1}^{N} {r}_{i}^{k} \qquad ( equation 6 )$$

TF Binding Site Prediction Via eRNA Presence

The receiver operating characteristic (ROC) curve is computed to quantify the ability of bidriectionality to predict TF ChIP binding. ENCODE-called peaks within a TF's ChIP-seq data are considered truth and randomly selected regions that do not overlap any previously seen ChIP-seq peak are considered a gold standard for noise. For each peak (truth or noise) a bidirectional model is fit using the expectation maximization algorithm. A Bayesian information criteria (BIC) score was calculated between the exponentially-modified Gaussian mixture model and a simple uniform distribution with support across the entire peak. A true positive is recorded if the BIC score exceeds a threshold i and the peak was one of the ENCODE peak calls. A false positive is recorded if the BIC score exceeds the threshold (τ) and the peak is a random noise interval. The threshold i is varied to obtain a ROC curve of and compute an area under the curve (AUC).

### Computation of Bimodality, ΔBIC

To assess whether the distribution of ChIP peaks or TF binding motif instances around an eRNA origin is bimodal, a pairwise distribution test was developed and employed. The ΔBIC score (in equation) is defined to be the difference in BIC scores between a single Laplace-Uniform mixture centered at zero (unimodal) and a two component Laplace-Uniform mixture with displacement away from 0, i.e. c (bimodal). The density function of a Laplace distribution with parameters (c,b) is provided in equation equation 7 and the formulation for the Uniform distribution of equation equation 2 is used.

$$p ( d ; c , b ) = \frac{1}{2 b} exp - \frac{\vert d - c \vert}{b} \qquad ( equation 7 )$$

Here D refers to the set of distances, dᵢ∈[−1500,1500], either the center of the TF binding peaks obtained from MACS or the center of TF binding motif instances from PSSM scanner relative to eRNA origin. If ΔBIC>>0, bimodality in TF peak location is assumed relative to the eRNA origin.

$${0}_{} ( D ; {Θ}^{*} ) = {\prod }_{i = 1}^{N} \frac{1}{3000} \\ {1}_{} ( D ; {Θ}^{*} ) = {\prod }_{i = 1}^{N} w \frac{1}{2 b} exp \{ - \frac{\vert {d}_{i} \vert}{b} \} + \frac{1 - w}{3000} \\ {2}_{} ( D ; {Θ}^{*} ) = {\prod }_{i = 1}^{N} \frac{w}{4 b} exp \{ - \frac{\vert {d}_{i} - c \vert}{b} \} + \frac{w}{4 b} exp \{ - \frac{\vert {d}_{i} + μ \vert}{b} \} + \frac{1 - w}{3000} \\ Δ BIC := - 2 ( log {( D )}_{1} - log {( D )}_{2} ) + k log ( \vert D \vert ) \qquad ( equation 8 )$$

τ* is optimized again by the Expectation Maximization algorithm where the update rules are given in equation.

$${d}_{t + 1} = \frac{1}{2 ( {r}^{a} + {r}^{b} )} ( {\sum }_{i = 1}^{n} {r}_{i}^{a} {d}_{i} + {\sum }_{i = 1}^{n} {r}_{i}^{a} {d}_{i} ) \\ {b}_{t + 1} = \frac{1}{2 ( {r}^{a} + {r}^{b} )} ( {\sum }_{i = 1}^{n} {r}_{i}^{a} \vert {d}_{i} \vert + {\sum }_{i = 1}^{n} {r}_{i}^{b} \vert {d}_{i} \vert ) \\ {w}_{t + 1} = \frac{{r}^{a} + {r}^{b}}{r} \\ {r}_{i}^{a} = \frac{p ( {d}_{i} ; c , b )}{p ( {d}_{i} ; c , b ) + p ( {d}_{i} ; - c , b ) + u ( {d}_{i} ; - 1500 , 1500 )} \\ {r}_{i}^{b} = \frac{p ( {d}_{i} ; - c , b )}{p ( {d}_{i} ; c , b ) + p ( {d}_{i} ; - c , b ) + u ( {d}_{i} ; - 1500 , 1500 )} \\ \begin{matrix} {r}_{i}^{u} = 1 - {r}_{i}^{a} + {r}_{i}^{b} & {r}^{x} = {\sum }_{i = 1}^{N} {r}_{i}^{x} & r = {r}^{a} + {r}^{b} + {r}^{u}\end{matrix} \qquad ( equation 9 )$$

A signal is referred to as bimodal when ΔBIC>500, estimated from the distribution in FIG. 5C.

### Motif Curation and Motif Scanning

Transcription factor binding motifs are summarized as a position specific probability distribution over the nucleotide (ATGC) alphabet, referred to commonly as a position weight matrix (PWM). These models were gathered from the HOCOMOCO database of hand-curated transcription factor binding motif models for human and mouse (downloaded from hocomoco.autosomesu/final_bundle/HUMAN/mono/). In total, there exist 641 and 427 motif models for human and mouse, respectively.

Motif scanning around bidirectionals was performed by the algorithm outlined by Staden (Staden: Searching for Motifs in Nucleic Acid Sequences, 93-102 (Springer New York, Totowa, N.J., 1994, which is hereby incorporated by reference in its entirety). False discovery rate (FDR) was quantified by the approach outlined by Storey (2007, The American Journal of Human Genetics, 80:502-09, which is hereby incorporated by reference in its entirety). Only sequences where the FDR did not exceed 10⁻⁶ were considered a significant TF-sequence motif instance and the center of the matching sequence was used for all subsequent analysis. The basic stationary background model was estimated from GC content of hg19 (human, 42.3%) and mm10 (mouse, 41.2%) genome builds. Motif scanning was implemented in the C++ programming language using the openMPI framework to perform massive parallelization on compute clusters. This implementation, referred to as MDS can be downloaded at biof-git.colorado.edu/dowelllab/MDS.

### MD-Level Hypothesis Testing

### The Motif Displacement Score

The Motif Displacement score (MD-level) relates the proportion of significant motif sites within some window 2*h divided by the total number of motifs against some larger window 2*H centered at all bidirectional origin events. It is calculated on a per PWM binding model basis.

Let Xⱼ={x₁, x₂, . . . } be the set of bidirectional origin locations genome wide for some experiment j. Let Yᵢ{y₁, y₂, . . . } be the set of all significant motif sites for some TF-DNA binding motif model i genome wide, which is static as it only depends on the genome build of interest. Therefore the set of all MD-levels is calculated by equation 10.

$$g ( {X}_{j} , {Y}_{i} ; a ) = {\sum }_{x ∈ {X}_{j}} {\sum }_{y ∈ {Y}_{i}} δ ( \vert x - y \vert < a ) \\ {md}_{j , i} = g ( {X}_{j} , {Y}_{i} ; h ) / g ( {X}_{j} , {Y}_{i} ; H ) \\ {md}_{j , i} ∈ [ 0 , 1 ) if h < H \qquad ( equation 10 )$$

Here, δ(⋅) is an indicator function that returns one if the condition (⋅) evaluates true otherwise to zero. The double sum, i.e. g(a), is naively O(X|X∥Y|) however data structures like interval trees reduce time to O(|X|log|Y|).

There exist 641 TF-DNA binding models in the HOCOMOCO database and therefore 641 MD-levels exist for some experiment j. Let mdᵢ be the MD-level computed for some TF-DNA binding motif model. Therefore let MDⱼ={md₁, md₂, . . . , md₆₄₁} be the vector of all MD-levels for some dataset j.

### MD-Level Significance Under Stationary Model

If yᵢ and xᵢ are uniformly distributed throughout the genome, i.e. following a homogeneous Poisson point process, then g(h) is distributed as a binomial distribution with parameters p,N (equation 11).

$$g ( h ) \sim B ( n , p ) \\ B ( k ; n , p ) = ( \begin{matrix}n \\ k\end{matrix} ) {( p )}^{k} {( 1 - p )}^{n - k} \\ where n = G ( H ) and p = h / H \qquad ( equation 11 )$$

In cases where g(H)>>0, the binomial is well approximated by a Gaussian distribution and hypothesis testing under some a level can proceed in the typical fashion. Significantly increased MD-levels (by a binomial test) is diagnostic of heightened motif frequency surrounding eRNA origins.

### MD-Level Significance Under a Non-Stationary Background Model

Motifs are not distributed uniformly throughout the genome. Specifically, particular regions of the genome, such as gene promoters, are known to exhibit significance sequence bias. Therefore, the localized GC content is highly non-stationary around some radius of size H. In the present study, it is assumed that H=1500 bps.

To control for this non-stationarity, a simulation based method is used to compute p-values for MD-levels under an empirical CDF, i.e. a localized background model. Let p be a 4×2H matrix where each column corresponds to a position from an origin and each row corresponds to a probability distribution over the DNA alphabet {A,C,G,T}. To be clear, p_{0,0} corresponds to the probability of an A at position —H from any bidirectional origin, similarly corresponds to the probability that a G occurs at exactly the point of the bidirectional p_{2,1500} origin.

Therefore, the simulation based method of the background model is simple. Given an experiment of Xⱼ bidirectional origin locations, |Xⱼ| sequences are simulated following this non-stationary GC content bias. Then, iterate over all PWM models and look for significant motif hits, as discussed in Motif Curation and Motif Scanning Summary statistics about the displacement of the motif relative to the set of synthetic sequences are then computed, i.e. MD={md₁, md₂, . . . , md₆₄₁}. In this dataset, any motif match is by complete chance alone. This process is iterated 10,000 times to compute a random distribution over mdᵢ, i.e. ^{→}mdᵢ, and thus it is possible to assess the probability of the observed (i.e. from real data) mdᵢ relative to the empirically simulated ^{→}mdᵢ.

### Cell Type and TF Enrichment Analysis

This section serves to outline the rational for determining if heightened MD-levels correlate with a specific cell type category. More traditional approaches such as a one-way ANOVA test (MD-levels computed from similar cell types are grouped and within group variance is assessed via a F-distribution) will not adequately account for MD-levels with little support (i.e. motif hits that overlap very few eRNAs). To overcome this, a method is presented that relies on performing hypothesis testing on all pairwise experimental comparisons.

Let j and k be two nascent transcription data sets of interest, then mds_{j,i} and mds_{k,i} refer to MD-levels for some TF-motif model (i) for hypothesis testing can be performed as outlined in the section entitled MD-level Hypothesis Testing. If α is the threshold at which mds_{j,i}-mds_{k,i} is considered to significantly increase, than it is expected on average α·N−1 false positives when considering a single experiment against the rest of the corpus of size N.

Put another way, if the random variable S_{j,i} refers to the number of times mds_{j,i}−mds_{k,i} is considered to significantly increase in a data set comparison then S_{j,i} is binomial distributed with parameters N−1 and a (equation 12), assuming that there is not a relationship between the motif model i and the experiment j.

$${S}_{j , i} = {\sum }_{k = 1}^{N} I ( p ( {mds}_{j , i} > {mds}_{k , i} ) < α ) \qquad ( equation 12 )$$

In practice a is set to 10⁻⁶ and I refers to an indicator function which returns 1 in the case where the statement evaluates to truth, otherwise 0.

Naively, we could now ask for all the data sets annotated as some cell type ct and then perform hypothesis testing on S_{ct} (the sum of S_{j,i}'s where experiment j belongs to the ct cell type set). Importantly, only data set pairs are considered for which i and j belong to different cell type sets. A single experiment within the cell type set might show strong association with a TF (i.e. 90% of the N−1 comparisons significantly deviate from zero) where the rest of the cell types show small numbers of significant deviations. By a binomial test, this is unlikely—even when considering the expansion induced by the cell type set—but intuitively does not fit into the notion of cell type association.

To this end, a final random variable A_{ct,i} is defined to be the number of times motif model i is significantly enriched for a data set j and that data set j belongs to some cell type (equation Error! Reference source not found.).

$$A = {\sum }_{j = 1}^{N} p ( {S}_{j , i} > S ) < {10}^{- 6} I ( j ∈ CT ) \qquad ( equation 13 )$$

where CT refers to the set of experiments that are annotated as cell type ct. From there A can be assessed across cell types and motif models under a contingency model using Fisher's exact test.
Transcription of the TF Gene when the MD-Level is Elevated or Depleted

To evaluate whether significantly altered (elevated or depleted) MD-levels reflect TF activity, the nascent transcription levels over the gene encoding the transcription factor are first calculated. To this end, all RefSeq genes were downloaded from hg19. Samples with less than 5,000 Tfit bidirectional regions were removed from subsequent consideration. FPKM was calculated for each gene in each human nascent transcription sample (n=491) over the body of the gene, defined here as 1 kb to the end of the gene. For all transcription factors in HOCOMOCO longer than 1 kb and with a RefSeq name (n=635 TFs), the maximum FPKM of all annotated isoforms was utilized. All transcription factor MD-levels were compared to expectation and classified on a per sample basis. Significant deviations from expectation were determined as passing both the stationary and non-stationary test (p-value<10⁻⁶)). TFs with significant deviation were subsequently labeled as elevated if they had a minimum MD-level of 0.1 and were above expectation, or depleted if they had a maximum MD-level of 0.1 and below expectation. To identify samples in which the TF is at expectation, a third set was labeled as at-expectation if they pass the stationary and non-stationary test (p-value<10⁻²). Across all samples, to avoid zero FPKM the minimum non-zero FPKM was utilized.

The Spearman's rank correlation coefficient and p-value across all samples (n=491; scipy v0.17.1) between MD-levels and the FPKM of the gene encoding the transcription factor was then calculated. When shuffling the FPKMs across samples, an average of 8.4 TFs is expected to show correlation (permutation testing 100 times, standard deviation 2.4 TFs). For all eRNAs (MD-level from non-promoter associated bidirectionals), 286 of 635 TFs show a correlation (p-value<0.01). For all bidirectionals (includes promoters), the same p-value cutoff finds 441 of 635 TFs with correlation (expectation 16.5, standard deviation 3.8).

Regions evaluated by a functional assay were then examined, namely CapStarr-seq, for their co-occurrence with eRNA origins. In CapStarr-seq mouse 3T3 cells were used, selected TF bound regions (by ChIP) and determined whether the bound regions functioned as an enhancer using a GFP expression assay. Identified regions were moved to mm10 coordinates using LiftOver. For comparison to nascent transcription, Tfit called bidirectionals (both eRNA and promoter origins) for mouse samples (SRR1233867, SRR1233868, SRR1233869, SRR1233870, SRR1233871, SRR1233872, SRR1233873, SRR1233874, SRR1233875, SRR1233876) from the 3T3 cell lines were combined. While 35.5% of regions classified as a strong enhancer (n=186) by CapStarr-seq contained a bidirectional origin only 7.9% of regions classified inactive (n=4406) had a bidirectional origin. Generally, bidirectionals within strong enhancers (by CapStarr-seq) were identified by Tfit in multiple nascent transcription replicates while bidirectionals within inactive regions were only in one nascent transcription replicate. Overall, regions defined as strong enhancers were 4× more likely to contain an eRNA origin than regions defined as inactive enhancers.

### MD-Level Significance Between Experiments

The MD-level constitutes a proportion and as long as h is upper bounded by H, then md_{j,i} will always exist within the semi-open interval [0,1). An important question is whether md_{j,i} has significantly shifted between two experiments j,k as a function of Xⱼ and Xₖ. This analysis is straightforward under the two proportion z-test. Specifically, the null and alternative hypothesis tests in equation 14 are tested.

H₀:md_{j,i}=md_{k,}i

H₁:md_{j,i}≠md_{k,}i(equation 14)

The pooled sample proportion (pᵢ) and standard error (SE) can then be computed as shown in equation 15. Therefore, test statistic z (equation 16) is normally distributed with mean 0 and variance 1.

$$\begin{matrix}{p}_{i} = \frac{( {md}_{j , i} \cdot g ( {X}_{j} , {Y}_{i} ; H ) + {md}_{k , i} \cdot g ( {X}_{k} , {Y}_{i} ; H ) )}{g ( {X}_{j} , {Y}_{i} ; H ) + g ( {X}_{k} , {Y}_{i} ; H )} & ( equation 15 ) \\ SE = \frac{p ( 1 - p )}{( 1 / g ( {X}_{j} , {Y}_{i} ; H ) + 1 / g ( {X}_{k} , {Y}_{i} ; H ) )} & \\ z = \frac{{md}_{j , i} - {md}_{k , i}}{\sqrt{SE}} \sim N ( 0 , 1 ) & ( equation 16 )\end{matrix}$$

Computation of the p-value can be assessed in the normal fashion under some a level. In all comparisons, multiple hypothesis correction outlined by Storey (2007) is used.

### Conditions Table

This study analyzed 771 nascent transcript datasets which span different organisms, cell types, treatments and conditions. To this end, a meta table .csv format is provided where each row corresponds to some nascent transcript dataset. The columns in this table proceed in this order: SRAnumber, organism, tissue, general_celltype specific_celltype, treatment_code, treated_or_like_treated, repnumber, keyword, exptype, mapped_reads, total_reads, percent_mapped, TSS, bidirectionals. SRAnumber provides the unique GEO identifier which was queried to pull down the original fastq files. Terms such as tissue, general_celltype, specific_celltype and treatment_code were populated by reviewing the publication associated with GEO SRA number. Fields such as mapped_reads, total_reads and percent_mapped refer to quality metrics output from running bowtie2. Finally, bidirectionals refer to the total number of bidirectional origins predicted by Tfit and TSS refers the proportion of those associated with a transcription start site (μ<1500 of RefSeq TSS annotation).

### Associated File Types

Two sets of data files are available: (1) a folder of Tfit predicted eRNA origins for the compendium of publicly available human and mouse nascent transcription data sets (771) (Supplemental Data 51); and (2) a histogram of motif locations surrounding eRNA origins for each of the 771 nascent transcript data sets and 641 motif models (Supplemental Data S2). These data files are available at dowell.colorado.edu/pubs/MDscore/

### Tfit Bidirectional Predictions

For each nascent transcription experiment, defined by SRR identifier number, Tfit was run using the default parameters discussed in Nascent Transcription Data Processing. Thusly, a comma separated file was generated for each dataset with four columns; chrom, start, stop, tss. The field chrom refers to the chromosome location of the bidirectional origin, the start and stop refer to the genomic location on that chromosome and tss will either return 1 or 0 depending on whether that bidirectional origin overlapped (μ<1500) a RefSeq transcriptional start site annotation. In this way, the set of eRNAs are those bidirectionals where tss=0.

Given that these FASTQ files were downloaded from the GEO database, each Tfit prediction file is uniquely named according to the specific SRR identifier from GEO. For example, a nascent transcription experiment from an estradiol time course experiment in MCF7 cells is SRR497920. Therefore the sites of bidirectional transcription by Tfit are in a file named: SRR497920.csv. All these files are located within the associated tar ball folder: “tfit_predictions” (downloadable at nascent.colorado.edu).

### Sites of TF Binding Motif Instances

At the time this study was conducted, 641 HOCOMOCO motif models exist. The genome was scanned for these nucleotide sites (as described in Motif Curation and Motif Scanning) and significant hits are represented in bed file format. In this way, the tar ball motif hits, associated with this study, contains 641 files each named by the HOCOMOCO motif model ID.

### Motif Displacement File Type

In addition to Tfit-predicted sites of bidirectional transcription, each experiment has an associated set of motif displacement histograms used to compute a wide array of statistics: MD-level, mean distance, etc. For each dataset in the conditions table, there exists a comma separated file where the first column refers to motif model ID from HOCOMOCO, the second column refers to whether or not this motif displacement distribution was computed using tss associated or non-tss associated Tfit bidirectional predictions. The final 3001 columns provide the position away from eRNA origin and the number of motifs observed at that position. This constitutes the empirically observed motif displacements histogram for the specified motif. Sites are considered a TF binding motif if the p-value using the PSSM from HOCOMOCO falls below 10⁻⁷.

Given that these FASTQ files were downloaded from the GEO database, each motif displacement file is uniquely named according to the specific SRR identifier from GEO. For example, a nascent transcription experiment from an estradiol time course experiment in MCF7 cells is SRR497920. Therefore the motif displacement file is named: SRR497920.csv. All these files are located within the associated tar ball folder: “Motif_Displacements” (downloadable at nascent.colorado.edu).

What is claimed is:

1. A laboratory method for evaluating the effect of a stimulus on a cell using a Motif-Displacement (MD) model that approximates transcription factor activity in the cell, the method comprising:
a) locating a first set of enhancer RNA (eRNA) origination sites in the cell's genomic DNA using a first genome-wide nascent transcription profile for the cell;
b) identifying DNA binding motif instances for transcription factors in the cell's genomic DNA;
c) for each eRNA origination site in the first set of eRNA origination sites, measuring a number of DNA binding motif instances for each of the transcription factors occurring within a first radius of the eRNA origination site and measuring a number of DNA binding motif instances for each of the transcription factors occurring within a second radius of the eRNA site, wherein the first radius and the second radius are each centered at each of the eRNA origination sites of the first set of eRNA origination sites, and the second radius is greater than the first radius;
d) calculating, using one or more processors executing instructions stored in a tangible, non-transitory storage medium, a first MD-level for each of the transcription factors based on the number of DNA binding motif instances for that transcription factor occurring within the first radius of the eRNA origination sites of the first set of eRNA origination sites and the number of DNA binding motif instances for that one transcription factors occurring within the second radius of the eRNA origination sites of the first set of eRNA origination sites;
e) applying a stimulus to the cell;
f) locating a second set of eRNA origination sites in the cell's genomic DNA using a second genome-wide nascent transcription profile for the cell, wherein the second genome-wide nascent transcription profile is generated after applying the stimulus to the cell;
g) for each eRNA origination site in the second set of eRNA origination sites, measuring a number of DNA binding motif instances for each of the transcription factors occurring within the first radius of the eRNA origination site and measuring a number of DNA binding motif instances for each of the transcription factors occurring within the second radius of the eRNA site;
h) calculating, using one or more processors executing instructions stored in a tangible, non-transitory storage medium, a second MD-level for each of the transcription factors based on the number of DNA binding motif instances for that transcription factor occurring within the first radius of the eRNA origination sites of the second set of eRNA and the number of DNA binding motif instances for that one transcription factors occurring within the second radius of the eRNA origination sites of the second set of eRNA origination sites; and
i) approximating effects of the stimulus on the transcription factor activity in the cell by identifying biologically significant differences between the first MD-level and the second MD-level.

2. The laboratory method according to claim 1, wherein the model approximates the effects of the stimulus on all transcription factors having a known DNA binding motif model, or a subset thereof.

3. The laboratory method according to claim 1, wherein the stimulus is a drug, a biologic, a compound or combination of compounds capable of initiating cellular differentiation or causing a disease state; an environmental stress, time, or a combination thereof.

4. The laboratory method according to claim 1, further comprising generating at least one of the first genome-wide nascent transcription profile for the cell and the second genome-wide nascent transcription profile.

5. The laboratory method according to claim 1 or claim 4, wherein the first genome-wide nascent transcription profile and the second genome-wide nascent transcription profile are each individually generated by a technique selected from: global run-on sequencing (GRO-seq), global run-on cap sequencing (GRO-cap), chromatin immunoprecipitation sequencing (ChIP-seq), precision nuclear run-on sequencing (PRO-seq), cap analysis of gene expression with deep sequencing (CAGE), 5′-end serial analysis of gene expression (SAGE), native elongating transcript sequencing (NET-seq), chromatin isolation by RNA purification (ChIRP-seq), assay for transposase-accessible chromatin with high throughput sequencing (ATAC-seq), transient transcriptome sequencing (TT-seq), and bromouridine UV sequencing (BruUV-seq).

6. The laboratory method according to claim 1, wherein the first set of eRNA origination sites and the second set of eRNA origination sites are each individually located utilizing one of: Tfit, dREG, groHMM, Vespucci, and FStitch.

7. The laboratory method according to claim 1, wherein the first radius is selected from between 50 base-pairs and 300 base-pairs.

8. The laboratory method according to claim 1, wherein the first radius is 150 base-pairs.

9. The laboratory method according to claim 1, wherein the second radius is selected from between 500 base-pairs and 3000 base-pairs.

10. The laboratory method according to claim 1, wherein the second radius is 150 base-pairs.

11. The laboratory method according to claim 1, wherein the second radius is 7 to 13 times larger than the first radius.

12. The laboratory method according to claim 1, wherein the second radius is 10 times larger than the first radius.

13. The laboratory method according to claim 1, wherein the first radius is 150 base-pairs and the second radius is 1500 base-pairs.

14. The laboratory method according to claim 1, wherein transcription factor activity for a given transcription factor is approximated as increased if the second MD-level is greater than the first MD-level, approximated as decreased if the second MD-level is smaller than the first MD-level, or approximated as unchanged if the second MD-level approximately equals the first MD-level.

15. A computer-based system for evaluating the effect of a stimulus on a cell using a Motif-Displacement (MD) model that approximates transcription factor activity in a cell, the system comprising:
one or more processors; and
a non-transitory, tangible storage medium containing instructions that, when executed by the processor, cause the one or more processors to:
a) locate a first set of enhancer RNA (eRNA) origination sites in the cell's genomic DNA using a first genome-wide nascent transcription profile for the cell;
b) identify DNA binding motif instances for transcription factors in the cell's genomic DNA;
c) for each eRNA origination site in the first set of eRNA origination sites, measuring a number of DNA binding motif instances for each of the transcription factors occurring within a first radius of the eRNA origination site and measuring a number of DNA binding motif instances for each of the transcription factors occurring within a second radius of the eRNA site, wherein the first radius and the second radius are each centered at each of the eRNA origination sites of the first set of eRNA origination sites, and the second radius is greater than the first radius;
d) calculate a first MD-level for each of the transcription factors based on the number of DNA binding motif instances for that transcription factor occurring within the first radius of the eRNA origination sites of the first set of eRNA origination sites and the number of DNA binding motif instances for that one transcription factors occurring within the second radius of the eRNA origination sites of the first set of eRNA origination sites;
e) locate a second set of eRNA origination sites in the cell's genomic DNA using a second genome-wide transcription profile for the cell, wherein the second genome-wide nascent transcription profile is generated after applying a stimulus to the cell;
f) for each eRNA origination site in the second set of eRNA origination sites, measuring a number of DNA binding motif instances for each of the transcription factors occurring within the first radius of the eRNA origination site and measuring a number of DNA binding motif instances for each of the transcription factors occurring within the second radius of the eRNA site,
g) calculate a second MD-level for each of the transcription factors based on the number of DNA binding motif instances for that transcription factor occurring within the first radius of the eRNA origination sites of the second set of eRNA origination sites and the number of DNA binding motif instances for that one transcription factors occurring within the second radius of the eRNA origination sites of the second set of eRNA origination sites; and
h) approximate effects of the stimulus on the transcription factor activity in the cell by identifying biologically significant differences between the first MD-level and the second MD-level.

16. The computer-based system according to claim 15, wherein the model approximates the effects of the stimulus on all transcription factors having a known DNA binding motif model, or a subset thereof.

17. The computer-based system according to claim 14, wherein the stimulus is a drug, a biologic, a compound or combination of compounds capable of initiating cellular differentiation or causing a disease state; an environmental stress, time, or a combination thereof.

18. The computer-based system according to claim 15, wherein the first genome-wide nascent transcription profile and the second genome-wide nascent transcription profile are each individually generated by a technique selected from: global run-on sequencing (GRO-seq), GRO-cap, chromatin immunoprecipitation sequencing (ChIP-seq), precision nuclear run-on sequencing (PRO-seq), cap analysis of gene expression with deep sequencing (CAGE), 5′-end serial analysis of gene expression (SAGE), native elongating transcript sequencing (NET-seq), chromatin isolation by RNA purification (ChIRP-seq), assay for transposase-accessible chromatin with high throughput sequencing (ATAC-seq), transient transcriptome sequencing (TT-seq), and bromouridine UV sequencing (BruUV-seq).

19. The computer-based system according to claim 15, wherein the first set of eRNA origination sites and the second set of eRNA origination sites are each individually located utilizing one of: Tfit, dREG, groHMM, Vespucci, and FStitch.

20. The computer-based system according to claim 15, wherein the first radius is selected from between 50 base-pairs and 300 base-pairs.

21. The computer-based system according to claim 15, wherein the first radius is 150 base-pairs.

22. The computer-based system according to claim 15, wherein the second radius is selected from between 500 base-pairs and 3000 base-pairs.

23. The computer-based system according to claim 15, wherein the second radius is 150 base-pairs.

24. The computer-based system according to claim 15, wherein the second radius is 7 to 13 times larger than the first radius.

25. The computer-based system according to claim 15, wherein the second radius is 10 times larger than the first radius.

26. The computer-based system according to claim 15, wherein the first radius is 150 base-pairs and the second radius is 1500 base-pairs.

27. The computer-based system according to claim 15, wherein transcription factor activity for a given transcription factor is approximated as increased if the second MD-level is greater than the first MD-level, approximated as decreased if the second MD-level is smaller than the first MD-level, or approximated as unchanged if the second MD-level approximately equals the first MD-level.

28. A processor-based method for identifying active transcription factors in a cell, the method comprising:
a) locating enhancer RNA (eRNA) origination sites in the cell's genomic DNA by analyzing a genome-wide nascent transcription profile for the cell;
b) identifying DNA binding motif instances for transcription factors in the cell's genomic DNA;
c) measuring a number of DNA binding motif instances for each of the transcription factors occurring within a first radius of each of the eRNA origination sites;
d) measuring a number of DNA binding motif instances for each of the transcription factors occurring within a second radius of each of the eRNA origination sites, wherein the first radius and the second radius are each centered at each of the eRNA origination sites and wherein the second radius is greater than the first radius;
e) using one or more processors to determine a Motif-Displacement (MD) model that approximates transcription factor activity in the cell, the processor executing instructions stored in a tangible, non-transitory storage medium in order to:
e1) calculate an observed MD-level for each of the transcription factors using a number of DNA binding motif instances for a transcription factor occurring within the first radius of the eRNA origination site and a number of DNA binding motif instances for that transcription factor occurring within the second radius of the eRNA origination site;
e2) calculate an expected MD-level for each of the transcription factors; and
e3) allocate each of the transcription factors as active in the cell if the calculated observed MD-level is greater than the expected MD-level and if the difference between the calculated MD-level and the expected MD-level is biologically significant.

29. The processor-based method according to claim 28, wherein the model determines the MD-level for each transcription factor having a known DNA binding motif model, or a subset thereof.

30. The processor-based method of claim 28, further comprising the step of identifying one or more compounds that are biologically effective with respect to the active transcription factors.

31. The processor-based method according to claim 28, further comprising generating the genome-wide nascent transcription profile for the cell.

32. The processor-based method according to claim 28 or claim 30, wherein the genome-wide nascent transcription profile is generated by a technique selected from: global run-on sequencing (GRO-seq), GRO-cap, chromatin immunoprecipitation sequencing (ChIP-seq), precision nuclear run-on sequencing (PRO-seq), cap analysis of gene expression with deep sequencing (CAGE), 5′-end serial analysis of gene expression (SAGE), native elongating transcript sequencing (NET-seq), chromatin isolation by RNA purification (ChIRP-seq), assay for transposase-accessible chromatin with high throughput sequencing (ATAC-seq), transient transcriptome sequencing (TT-seq), and bromouridine UV sequencing (BruUV-seq).

33. The processor-based method according to claim 28, wherein the eRNA origination sites are identified utilizing one of: Tfit, dREG, groHMM, Vespucci, and FStitch.

34. The processor-based method according to claim 28, wherein the first radius is selected from between 50 base-pairs and 300 base-pairs.

35. The processor-based method according to claim 28, wherein the first radius is 150 base-pairs.

36. The processor-based method according to claim 28, wherein the second radius is selected from between 500 base-pairs and 3000 base-pairs.

37. The processor-based method according to claim 28, wherein the second radius is 150 base-pairs.

38. The processor-based method according to claim 28, wherein the second radius is 7 to 13 times larger than the first radius.

39. The processor-based method according to claim 28, wherein the second radius is 10 times larger than the first radius.

40. The processor-based method according to claim 28, wherein the first radius is 150 base-pairs and the second radius is 1500 base-pairs.
